(19) United States
(12) Patent Application Publication
(10) Pub. No.: US 2025/0281294 A1
(43) Pub. Date: Sep. 11, 2025

(54) FLOW RESTRICTORS FOR BLOOD VESSELS

(71) Applicant: EDWARDS LIFESCIENCES CORPORATION, Irvine, CA (US)

(72) Inventors: Gideon Meyer-Brodnitz, Ramot Menashe (IL); Roy Shitrit, Zichron Yaakov (IL); Yehoshua Strauss, Elkana (IL); Roei Mayo, Irvine, CA (US); Yaeer E. Lev, Torrance, CA (US)

(73) Assignee: EDWARDS LIFESCIENCES CORPORATION, Irvine, CA (US)

(21) Appl. No.: 19/215,215

(22) Filed: May 21, 2025

Related U.S. Application Data
(63) Continuation of application No. PCT/US2023/084009, filed on Dec. 14, 2023.
(60) Provisional application No. 63/476,132, filed on Dec. 19, 2022.

(51) Int. Cl. A61F 2/24

(52) U.S. Cl. CPC A61F 2/2475; A61F 2250/0013

(57) ABSTRACT

A flow restrictor for a blood vessel can include a frame positionable within a blood vessel, and a leaflet or flap comprising an inflow end and an outflow end. The inflow end of the leaflet or flap can be configured to move (optionally using a control element) in response to a first increase in a blood pressure within the blood vessel, and in response to the blood pressure being within a first blood pressure range. The flow restrictor can be further configured to be a bi-modal flow restrictor, wherein the leaflet or flap can be configured to collapse or prolapse in response to the blood pressure being within a second blood pressure range. In some cases, the bi-modal flow restrictor can include an inner valve configured to move with respect to the frame in response to the blood pressure being within the second blood pressure range.

### CROSS-REFERENCE TO RELATED APPLICATIONS

This application is a continuation of International Application Ser. No. PCT/US2023/084009, filed Dec. 14, 2023, which claims the benefit of U.S. Provisional Patent Application Ser. No. 63/476,132, filed Dec. 19, 2022, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of medical devices and procedures, and more specifically to the field of blood flow management in blood vessels.

### BACKGROUND

Chronic kidney disease (CKD) is a common comorbidity with many patients who suffer from chronic Heart Failure (HF). HF patients may also have elevated right atrium pressure, which may impair kidney function. In HF patients with elevated right atrium pressure, the kidneys may attempt to perform a diuresis process, but such a process may be difficult to perform efficiently due to the elevated pressure. For example, elevated right atrium pressure may hinder the ability of the kidneys to drive forward the flow of blood for accomplishing proper and efficient diuresis. Such unbalanced pressure coupled with the typical poor kidney efficiency of CKD patients may lead to an unending cycle of fluid overload for a person, which may result in an increase in congestion and heart failure admissions to the hospital.

### SUMMARY

Described herein are one or more methods and/or devices to facilitate management of blood flow through and/or into one or more blood vessels and/or chambers of a heart. There is a need for new and useful systems and methods for flow restrictors for blood vessels. In particular, there is a need for systems, devices, and methods that enable modulating and/or balancing of blood flow through a blood vessel, for example, to occlude, partially occlude, and/or otherwise manage or regulate blood flow to or through a portion of a blood vessel.

In some embodiments, a flow restrictor for a blood vessel, includes a frame positionable within a blood vessel, and two or more leaflets each comprising an inflow end and an outflow end. The outflow end of each leaflet can be coupled to the frame, and the inflow end of each leaflet can be movable in a radial direction, wherein the radial direction is approximately perpendicular to a central axis of the frame. Each leaflet can define a cavity having an opening that is configured to face a direction of blood flow within the blood vessel. The inflow end of each leaflet can be configured to move toward each other in response to a first increase in a blood pressure within the blood vessel, and in response to the blood pressure within the blood vessel being within a first blood pressure range.

In some embodiments, a bi-modal flow restrictor for a blood vessel, includes: an outer frame positionable within a blood vessel; an inner valve positioned within the outer frame; two or more leaflets each comprising an inflow end and an outflow end; and a first spring positioned between the inner valve and the outer frame. The inner valve can be movable in an axial direction within the outer frame, and the axial direction is approximately parallel with a central axis of the outer frame. The outflow end of each leaflet can be coupled to the inner valve, the inflow end of each leaflet can be movable in a radial direction, wherein the radial direction is approximately perpendicular to the central axis of the outer frame. Each leaflet can define a cavity having an opening that is configured to face a direction of blood flow within the blood vessel, and the inflow end of each leaflet can be configured to move toward each other in response to a first increase in a blood pressure within the blood vessel, and in response to the blood pressure within the blood vessel being within a first blood pressure range. The first spring can be configured to hold the inner valve in a first axial position within the outer frame when the blood pressure within the blood vessel is within the first blood pressure range, and to allow the inner valve to move to a second axial position when the blood pressure within the blood vessel is within a second blood pressure range, wherein the second axial position is downstream from the first axial position. A lower limit of the second blood pressure range can be higher than an upper limit of the first blood pressure range. An outflow ring of the inner valve can be pressed against a seat of the outer frame when the inner valve is in the first axial position, and the outflow ring of the inner valve can be configured to move away from the seat of the outer frame when the inner valve is in the second axial position thereby allowing the blood flow to bypass the inner valve and flow between the inner valve and the outer frame.

In some embodiments, a bi-modal flow restrictor for a blood vessel, includes an outer docking frame positionable within a blood vessel, and an inner valve positioned within the outer docking frame. The inner valve can be configured to be sealed against the outer docking frame when blood pressure within the blood vessel is within a first blood pressure range. The inner valve can include two or more leaflets configured to move closer to each other in response to elevated pressure within the blood vessel and restrict flow through the inner valve when the inner valve is sealed against the outer docking frame. The inner valve and the outer docking frame can be configured such that a gap is formed between the inner valve and the outer docking frame when blood pressure within the blood vessel is within a second blood pressure range, wherein a lower limit of the second blood pressure range is higher than an upper limit of the first blood pressure range. Blood can flow through the gap between the inner valve and the outer docking frame when the gap is formed.

In some embodiments, a flow restrictor for a blood vessel, includes: a frame positionable within a blood vessel; a first flap comprising a first flap inflow end and a first flap outflow end; a first control element coupled to the first flap; a first helical tube coupled to a wall of the blood vessel; and a first guide tube coupled to the frame. The first flap inflow end can be coupled to the frame such that the first flap inflow end is movable in a radial direction, wherein the radial direction is approximately perpendicular to a central axis of the frame. The first control element can be fixed to a first end of the first helical tube, can extend through the first helical tube, can exit the first helical tube at a second end, can pass through the first guide tube, and can be fixed to the first flap outflow end. The first helical tube can be configured to expand in response to an enlargement of the blood vessel, and the first control element can be configured to pull the first flap radially inward in response to the expansion of the first helical tube.

In some embodiments, a flow restrictor for a blood vessel, includes: a frame configured to be coupled to a wall of a blood vessel; a first flap coupled to an outflow end of the frame using a first coupling such that the first flap is movable about the first coupling; and a first member coupled to an inflow end of the frame and to the first flap. A cross-section of the frame approximately perpendicular to a central axis of the frame can be approximately oval-shaped. The first member can have an approximately constant length. In response to an increase in blood pressure, the cross-sectional area of the frame can be configured to increase by assuming a less oval cross-sectional shape approximately perpendicular to the central axis of the frame, and the first member can be configured to move the first flap in response to the frame assuming the less oval cross-sectional shape.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 shows an example of a flow restrictor including leaflets, in accordance with some embodiments.

FIG. 2 shows an example of a flow restrictor including flexible commissures, in accordance with some embodiments.

FIGS. 3A-3C show an example of a bi-modal flow restrictor operating in three different blood pressure ranges, in accordance with some embodiments.

FIGS. 4A-4C show an example of a flow restrictor operating in the three different blood pressure ranges, in accordance with some embodiments.

FIG. 5 shows a cross-sectional side view schematic of an example of a flow restrictor within a blood vessel, in accordance with some embodiments.

FIGS. 6A-6C show cross-sectional side view schematics of an example of a flow restrictor with a bi-modal flow restriction assembly within a blood vessel, in accordance with some embodiments.

FIG. 7 shows an example of a cross-sectional side view schematic of a bi-modal flow restrictor with a bi-modal flow restriction assembly within a blood vessel, in accordance with some embodiments.

FIG. 8 shows an example position of a bi-modal flow restrictor within the inferior vena cava (IVC) of a human patient, in accordance with some embodiments.

FIGS. 9A-9F show stages of deployment and utilization of a bi-modal flow restrictor, in accordance with some embodiments.

FIGS. 10A-10B show a cut-away side view and an isometric view, respectively, of an example of a bi-modal flow restrictor similar to that shown in FIGS. 7-9F, in accordance with some embodiments.

FIGS. 11A-11H show isometric and cut-away views, from different angles and with different components removed to show the assemblies, of an example of a bi-modal flow restrictor similar to that shown in FIGS. 7-9F, in accordance with some embodiments.

FIGS. 12A-12B show isometric views from different angles of an inner valve of an example of a bi-modal flow restrictor similar to that shown in FIGS. 7-9F, in accordance with some embodiments.

FIGS. 13A-13C show isometric views from different angles of an outer frame of an example of a bi-modal flow restrictor similar to that shown in FIGS. 7-9F, in accordance with some embodiments.

FIGS. 14A-14B show isometric views from different angles of an example of a bi-modal flow restrictor similar to that shown in FIGS. 7-9F, in accordance with some embodiments.

FIGS. 15A-15D show isometric views and a top-down view (in FIG. 15B) of an example of a flow restrictor that can be positioned within a blood vessel, in accordance with some embodiments.

FIGS. 16A and 16B show an example of flow restrictor with one flap in a relatively extended and in a relatively retracted position, respectively, in accordance with some embodiments.

FIGS. 17A-17C show isometric views of an example flow restrictor that includes one or more flaps attached to, and movable by, a rigid push member, in accordance with some embodiments.

FIGS. 18A-18C show top-down views of the flow restrictor in FIGS. 17A-17C, in states that correspond to those shown in FIGS. 17A-17C, respectively, in accordance with some embodiments.

FIGS. 19A-19C show isometric views of an example flow restrictor, in accordance with some embodiments.

FIGS. 20A-20C show top-down views of an example flow restrictor, in accordance with some embodiments.

FIGS. 21A-21C show isometric views of an example flow restrictor, in accordance with some embodiments.

FIGS. 22A-22C show top-down views of an example flow restrictor, in accordance with some embodiments.

FIGS. 23A-23C show isometric views of an example flow restrictor, in accordance with some embodiments.

FIGS. 24A-24C show top-down views of an example flow restrictor, in accordance with some embodiments.

FIGS. 25A-25C show isometric views of an example flow restrictor, in accordance with some embodiments.

FIGS. 26A-26C show top-down views of an example flow restrictor, in accordance with some embodiments.

FIGS. 27A-27C show isometric views of an example flow restrictor, in accordance with some embodiments.

FIGS. 28A-28C show top-down views of an example flow restrictor, in accordance with some embodiments.

FIG. 29 shows a flowchart of an example method of restricting blood flow within a blood vessel, in accordance with some embodiments.

FIG. 30 shows a flowchart of an example method of restricting blood flow within a blood vessel, in accordance with some embodiments.

FIG. 31 shows a flowchart of an example method of restricting blood flow within a blood vessel, in accordance with some embodiments.

FIG. 32 shows a flowchart of an example method of restricting blood flow within a blood vessel, in accordance with some embodiments.

FIG. 33 shows a flowchart of an example method of restricting blood flow within a blood vessel, in accordance with some embodiments.

FIG. 34 shows a flowchart of an example method of restricting blood flow within a blood vessel, in accordance with some embodiments.

FIG. 35 shows a flowchart of an example method of deploying a flow restrictor within a blood vessel, in accordance with some embodiments.

FIG. 36 illustrates a schematic representation of portions of a subject that may include a flow modulating device implanted therein.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology may now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the systems and methods described herein may enable modulating and/or balancing of blood flow through a blood vessel. The modulating and/or balancing of blood flow may be performed by the devices described herein to occlude, partially occlude, and/or otherwise manage or regulate blood flow to or through a portion of a blood vessel. In some examples, such modulation and/or balancing of blood flow to or through a blood vessel may result in additionally modulating pressure in the right atrium of the heart and/or other organs of the body.

The examples presented herein may relate to providing devices, methods, and/or methods of treatment (MOTs) for modulating and/or otherwise managing blood flow to or through particular blood vessels. The terminology of restricting blood flow, regulating blood flow, modulating blood flow, managing blood flow, and balancing blood flow cause regulation of blood pressure, modulation of blood pressure, management of blood pressure, and/or balancing of blood pressure. As such, for example, a flow modulation device is synonymous with a pressure regulating device (i.e., a flow regulator is synonymous with a pressure regulator). In some examples, the devices described herein may include blood flow management devices for reducing blood flow through a blood vessel, such as the Superior Vena Cava (SVC) and the Inferior Vena Cava (IVC), or related vessels. Managing blood flow through the SVC or IVC can be achieved by the devices described herein to provide an advantage of improving perfusion of the kidneys. In particular, the devices described herein may generate a pressure gradient across the kidneys by decreasing central venous pressure by restricting, balancing, or otherwise modifying particular blood flow through the SVC and/or IVC, resulting in improved kidney perfusion and function.

In some examples, the devices, methods, and/or MOTs described herein may be utilized to solve a technical problem of unwanted pressure increases in the right atrium in patients that have chronic kidney disease (CKD) and/or heart failure (HF). For example, patients with CKD and/or HF may exhibit reduced kidney function when pressure in the right atrium of the heart is above a predefined pressure threshold. The predefined pressure threshold may be used as a basis to determine whether a patient is exhibiting low vessel pressure (e.g., below the predefined pressure threshold) or high vessel pressure (e.g., above the predefined pressure threshold). When vessel pressure is determined to be high, the devices, methods, and/or MOTs can provide a technical solution to the technical problem recited above. For example, each of the devices described herein may be used to decrease pressure within one or more vessels to avoid right atrium pressure increases and/or pressure variations. In particular, the devices, methods, and/or MOTs described herein can be used to reduce and maintain low pressure in the right atrium, which provides a technical effect of enabling the kidneys to more effectively filter blood.

In addition, the devices, methods, and/or MOTs described herein can solve a further technical problem of accumulation of blood in the venous system. For example, the devices described herein may be used to reduce the accumulation of blood in the venous system, which can provide an advantage and technical effect of ensuring that pressure is not increased in the IVC. Such devices can advantageously eliminate excessive hospital readmissions and/or can provide for a long-term blood flow management therapy, improving both quality of life and overall survival rates and with a lower cost to a healthcare system.

Furthermore, the devices, methods, and/or MOTs described herein can be used to solve a further technical problem of regulating blood flow return, thus further mitigating pressure build-up in the right atrium. The examples described herein can perform blood flow management actively and/or passively to assist in reducing and/or maintaining right atrium pressures to a relatively low pressure even when a surge in blood volume occurs in one or more vessels of the venous system.

In some examples, the devices, methods, and/or MOTs described herein can be used to solve a further technical problem of exertion-related blood pressure in patients that have a flow restrictor implanted within a blood vessel. For example, flow restrictor devices described herein can be configured to restrict blood flow through a blood vessel at elevated blood pressures (e.g., from about 10 mmHg to about 25 mmHg, or from about 15 mmHg to about 25 mmHg), and also be configured to advantageously permit a larger amount of blood flow (by not restricting or minimally restricting blood flow through the blood vessel) at exertion-related blood pressures (e.g., greater than 25 mmHg, or greater than 30 mmHg). In some cases, the flow restrictors described herein can be configured to permit a larger amount of blood flow through the blood vessel at exertion-related blood pressures (e.g., greater than 25 mmHg, or greater than 30 mmHg), which can advantageously provide a patient with sufficient blood flow through the blood vessel during periods of exercise or stress to prevent negative side effects (e.g., fainting).

In some cases, patients who suffer from congestive heart failure (CHF) can also experience impaired renal function, as impaired renal function can be caused by increased systemic venous congestion as a result of low cardiac output and low blood pressure. The renal pressure gradient (between the renal arteries and renal veins) may be decreased due to elevated renal venous pressure, lowering glomerular filtration rate (GFR), which is the rate at which the kidney filters blood, for example, below 90 mL/min, which can be indicative of chronic kidney disease (CKD) that may eventually lead to end stage renal failure. Thus, reduction of renal venous pressure may improve GFR and reduce blood volume retention. Nevertheless, it may be desirable for a device (e.g., a flow restrictor described herein) configured to limit central venous volume to operate in a bi-modal fashion, configured to reduce venous pressure when the patient is at rest (e.g., under normal or elevated blood pressure ranges), yet allow undisturbed or minimally disturbed venous flow when the patient exercises (or experiences an exertion-related blood pressure), so as to meet the dynamic flow/pressure requirements. Moreover, any such solution, when provided as an implantable device, can be percutaneously deliverable and can operate in a manner that minimizes risk of thrombosis.

Disclosed herein are systems and methods for a flow restrictor for a blood vessel. In some examples, the implantable flow modulating devices (or flow restrictors) described herein may be used in blood flow occlusion therapy. For example, the devices described herein may relate to venous occlusion therapy using implantable and/or electronically controlled flow restricting devices for the treatment of acute heart failure. Some devices may be non-implantable or partially implantable. Some devices operate without any powered input, and are triggered by changes in anatomy and/or changes in physiology.

### Systems and Devices

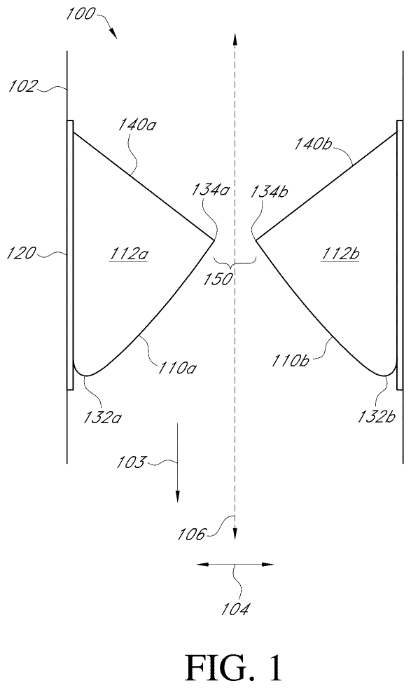

FIGS. 1-28A show examples of flow modulating devices that are flow restrictors for blood vessels. The flow restrictors described herein can be used to occlude, partially occlude, and/or otherwise manage or regulate blood flow to or through a portion of a blood vessel, for example, to reduce blood pressure in blood vessels and/or organs (e.g., kidneys) downstream from the flow restrictor. The flow restrictors described herein can additionally or alternatively be used for any suitable applications, clinical or otherwise. For example, the flow restrictors described herein can be configured and/or adapted to function for any suitable application requiring restriction of blood flow through or within a blood vessel, for example, to increase blood flow through an organ (e.g., the kidneys), to modulate pressure in the right atrium of the heart, and/or to reduce the accumulation of blood in the venous system.

In some examples, flow restrictors for blood vessels include inwardly extendable leaflets. In some examples, flow restrictors include leaflets forming pocket-like configurations, wherein the inflow ends of the leaflets are attached to springs (coupled to the anchoring frame) or to flexible commissures, allowing the leaflets to move radially inward (closer to each other) in response (optionally, proportional to) elevated venous pressure. These flow restrictors can provide a mechanically driven solution by which glomerular filtration rate (GFR, a metric of kidney function) can be improved in CKD patients, as an alternative (or in lieu of) conventional pharmaceutical treatments.

FIG. 1 is a side view schematic of an example of a flow restrictor 100 with inwardly extendable leaflets 110a, 110b. The leaflets 110a, 110b form cavities 112a, 112b (or pocket-like configurations) facing the blood flow. The blood flows in blood vessel 102 in direction 103. The blood flow enters the cavities 112a, 112b and applies pressure to the leaflets 110a, 110b, which are configured to move (or extend) toward each other in response to the blood flow.

FIG. 1 shows an example of a flow restrictor 100 that includes leaflets 110a, 110b coupled along their outflow ends 132a, 132b to the outflow end of a frame 120 (deployed within a blood vessel 102), having their inflow ends 134a, 134b movable in the radial direction 104, toward and away from the inflow end of frame 120. The radial direction 104 is approximately perpendicular to a central axis 106 (along an axial direction) of frame 120. The leaflets 110a, 110b can be configured to bias the inflow ends 134a, 134b of the leaflets 110a, 110b radially (in radial direction 104) outward, closer to the frame, in a free state thereof (or under low (or normal) blood pressure conditions). In some examples, the inflow ends 134a, 134b of the leaflets 110a, 110b are coupled to the frame 120 via springs 140a, 140b that are configured to bias the leaflets 110a, 110b radially (in radial direction 104) outward, closer to the frame, in a free state thereof (or under low (or normal) blood pressure conditions). Cavities 112a, 112b face the direction of blood flow. The outflow ends 132a, 132b of leaflets 110a, 110b can be directly attached to frame 120, and the inflow ends 134a, 134b are spaced away (radially inward) from the frame, thereby forming cavities 112a, 112b against which blood flow can impact.

As the blood flow propagates downstream in blood vessel 102 in FIG. 1, it can impinge against (or apply a force, or pressure to) the leaflets 110a, 110b in a manner that causes their inflow ends 134a, 134b to move closer toward each other (in radial direction 104), extending optional springs 140a, 140b, thereby narrowing the open area 150 (or lumen) through which blood can flow. The balance between the spring constant and the blood pressure can be selected such that leaflets 110a, 110b move farther inward (toward each other) in proportion with an increase in blood pressure, such that the higher the pressure is (at the inflow side), the narrower is the open area 150 (or lumen) through which blood may flow. In some examples, springs 140a, 140b are not present, and leaflets 110a, 110b include mechanical properties (e.g., stiffness) that enable their inflow ends 134a, 134b to move closer toward each other (in radial direction 104) in response to an increase in blood flow (or an increase in blood pressure), and then to return to their initial positions under low (or normal) blood pressure conditions.

In some examples, flow restrictor 100 can be positioned within the inferior vena cava or other blood vessels, such as upstream from the renal veins. Increased venous pressure within the blood vessel can result in greater restriction of blood flow through the flow restrictor 100, thereby lowering blood pressure downstream from the leaflets 110a, 110b. For example, flow restrictor 100 can be positioned within a blood vessel upstream from the renal veins, which can decrease the venous pressure at the renal veins, eventually increase renal pressure gradient, and improve kidney filtration rates.

In some examples, flow restrictor 100 can be designed to (or be configured to) allow leaflets 110a, 110b to prolapse in the downstream direction when a venous pressure exceeds a threshold, thereby allowing unrestricted (or minimally restricted) blood flow through flow restrictor 100. For example, blood pressure within blood vessel 102 may exceed a threshold due to exertion (e.g., exercise induced, or stress induced). In some examples, flow restrictor 100 is configured such that leaflets 110a, 110b prolapse in the downstream direction in response to exertion-related blood pressure (e.g., due to exercise or stress) that exceeds a blood pressure threshold. After the blood pressure falls back below the threshold, then leaflets 110a, 110b (and optionally springs 140a, 140b) can be configured to revert to the configuration wherein cavities 112a, 112b are formed with openings facing the direction of the blood flow. In such cases, when the blood pressure is lowered compared to exertion-related blood pressure conditions, leaflets may cease to be prolapsed causing the inflow ends 134a, 134b of leaflets 110a, 110b to move closer to one another in the radial direction 104 (optionally due to springs 140a, 140b recompressing) and causing the open area 150 (or lumen) to decrease to its former size.

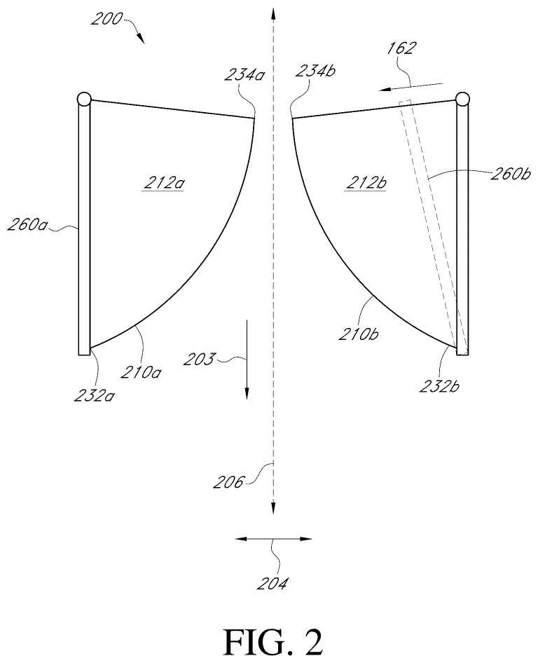

FIG. 2 shows an example of a flow restrictor 200 including flexible commissures in which the inflow ends 234a, 234b and the outflow ends 232a, 232b of leaflets 210a, 210b are coupled to flexible commissures 260a, 260b, which are in turn coupled to a frame (not shown) at their outflow ends. As in the previous example, leaflets 210a, 210b form cavities 212a, 212b (or pocket-like configurations) with openings facing the direction of the blood flow. The blood flows in a blood vessel in direction 203. The blood flow enters cavities 212a, 212b and applies pressure to leaflets 210a, 210b, which are configured to move (or extend) toward each other in the radial direction in response to the blood flow.

The commissures 260a, 260b of flow restrictor 200 can be biased to assume a straight vertical configuration (parallel to central axis 206 of a frame (not shown) of flow restrictor 200, for example), with their inflow ends 234a, 234b bendable radially (in direction 204, which is approximately perpendicular to central axis 206) inward when elevated pressure of the blood flow impinging against leaflets 210a, 210b, exerts a force sufficient to bend flexible commissures 160a, 160b and move leaflets 210a, 210b closer to each other, thereby restricting blood flow through flow restrictor 200.

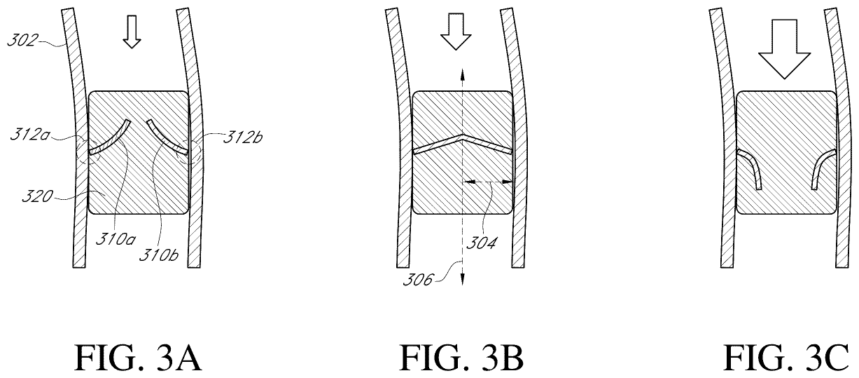

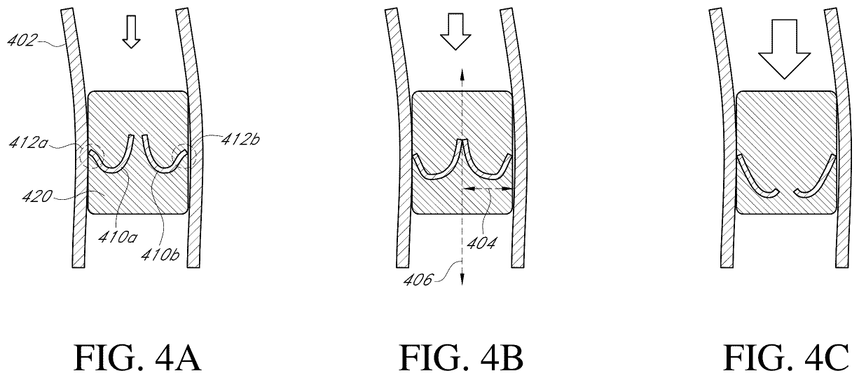

FIGS. 3A-4C show examples of bi-modal flow restrictors similar to those shown in FIGS. 1 and 2, with leaflet configurations designed to restrict blood flow in response to elevated blood pressure. In some cases, the bi-modal flow restrictors shown in FIGS. 1-2, 3A-3C, and 4A-4C restrict blood flow in proportion to elevated blood pressure during normal activity of the patient, and allow blood to flow therethrough with minimal obstruction during exertion-related elevated pressure (e.g., due to exercise activity and/or stress). In some cases, the bi-modal flow restrictors shown in FIGS. 1-2, 3A-3C, and 4A-4C restrict blood flow in proportion to elevated blood pressure in a first blood pressure range (e.g., from about 10 mmHg to about 25 mmHg, or from about 15 mmHg to about 25 mmHg), and allow blood to flow therethrough with minimal obstruction in a second blood pressure range, where the second blood pressure range is greater than a threshold blood pressure (e.g., about 25 mmHg or about 30 mmHg).

FIGS. 3A-3C show a first example of a bi-modal flow restrictor operating in three different blood pressure ranges. FIGS. 4A-4C show a second example of a flow restrictor operating in the three different blood pressure ranges. FIGS. 3A and 4A show flow restrictors operating in a normal blood pressure range (e.g., from about 1 mmHg to about 15 mmHg, or from about 2 mmHg to about 8 mmHg, or from about 5 mmHg to about 15 mmHg), wherein the inflow ends of the leaflets of the flow restrictor are in a first state (or initial state, or normal state) defining an open area through which blood can flow through the flow restrictor. FIGS. 3B and 4B show flow restrictors operating in an elevated blood pressure range (e.g., from about 10 mmHg to about 25 mmHg, or from about 15 mmHg to about 25 mmHg), wherein the inflow ends of the leaflets of the flow restrictor have moved towards one another reducing the open area through which blood can flow. FIGS. 3C and 4C show flow restrictors operating in an exertion-related blood pressure range (e.g., greater than about 25 mmHg, or greater than about 30 mmHg), wherein the leaflets have prolapsed (or collapsed) and the inflow ends of the leaflets of the flow restrictor have moved away from one another increasing the open area through which blood can flow.

The example bi-modal flow restrictor in FIGS. 3A-3C utilizes relatively short and relatively rigid leaflets 310a, 310b compared to the example flow restrictor in FIGS. 4A-4C, which utilizes relatively elongated and relatively soft (or relatively flexible) leaflets 410a, 410b.

The example bi-modal flow restrictor in FIGS. 3A-3C utilizes relatively short and relatively rigid leaflets 310a, 310b coupled to frame 320 to restrict blood flow in response to elevated blood pressure (e.g., due to pulmonary overload). Leaflets 310a, 310b are coupled to frame 320 at coupling regions 312a, 312b. Leaflets 310a, 310b are relatively short, such that when they extend towards one another from the edge of the frame 320 they meet to close the open space between them only when they are relatively straight (or do not have much curvature), as shown in FIG. 3B. In some examples, a length of leaflets 310a, 310b can be shorter than a radius 304 of the frame 320, wherein the radius is measured from a central axis 306 to the edge of frame 320 (or to coupling regions 312a, 312b). Leaflets 310a, 310b are relatively rigid compared to the coupling regions 312a, 312b. Therefore, when leaflets 310a, 310b move, due to a change in blood pressure, the leaflets 310a, 310b deform less than coupling regions 312a, 312b, as shown in FIGS. 3A-3C. For example, in FIG. 3C the coupling regions have flexed to allow the leaflets to prolapse. The leaflets in the flow restrictor in FIG. 3C are also flexed and show an opposite curvature to the leaflets under normal blood pressure conditions in FIG. 3A.

FIG. 3A shows the bi-modal flow restrictor under normal blood pressure conditions (e.g., about 1 mmHg to about 15 mmHg), where leaflets 310a, 310b are spaced apart such that they are, for example, about 60% to about 80% closed (meaning that the open area between the inflow ends of leaflets 310a, 310b includes about 40% to about 20% of the total cross-sectional area of the lumen of the flow restrictor, under normal blood pressure conditions). The leaflets 310a, 310b can be spaced apart such that they are from about 20% to about 80% closed (meaning that the open area between the inflow ends of leaflets 310a, 310b includes about 80% to about 20% of the total cross-sectional area of the lumen of the flow restrictor, under normal blood pressure conditions).

FIG. 3B shows the bi-modal flow restrictor under elevated blood pressure conditions, where the blood pressure in blood vessel in FIG. 3B is higher than the blood pressure in the blood vessel 302 in FIG. 3A (e.g., from about 10 mmHg to about 15 mmHg, or from about 15 mmHg to about 25 mmHg). Leaflets 310a, 310b in FIG. 3B are spaced closer together (or touching) such that they are, for example, about 90% to about 100% closed (meaning that the open area between the inflow ends of leaflets 310a, 310b includes about 10% to about 0% of the total cross-sectional area of the lumen of the flow restrictor, under the elevated blood pressure conditions). In general, under elevated blood pressure conditions, leaflets 310a, 310b can be spaced closer together (or touching) such that they are about 60% to about 100% closed (meaning that the open area between the inflow ends of leaflets 310a, 310b includes about 40% to about 0% of the total cross-sectional area of the lumen of the flow restrictor, under the elevated blood pressure conditions). Leaflets 310a, 310b closing in FIG. 3B reduces the amount of blood that can flow through the flow restrictor in this example, thereby lowering blood pressure downstream from leaflets 310a, 310b.

FIG. 3C shows the bi-modal flow restrictor under exertion-related blood pressure conditions, where the blood pressure in blood vessel 302 in FIG. 3C is higher than the blood pressure in the blood vessel in FIGS. 3A and 3B (e.g., greater than about 25 mmHg, or greater than about 30 mmHg). Leaflets 310a, 310b in FIG. 3C are prolapsed (wherein leaflets 310a, 310b move in the downstream direction) such that they are, for example, about 80% to about 100% opened (meaning that the open area between the inflow ends of leaflets 310a, 310b includes about 80% to about 100% of the total cross-sectional area of the lumen of the flow restrictor, under the exertion-related blood pressure conditions). In general, under exertion-related blood pressure conditions, leaflets 310a, 310b in FIG. 3C are prolapsed (e.g., leaflets 310a, 310b move in the downstream direction) such that they can be about 5% to about 100% (or about 10% to about 90%) opened (meaning that the open area between the inflow ends of leaflets 310a, 310b includes about 5% to about 100% of the total cross-sectional area of the lumen of the flow restrictor, under the exertion-related blood pressure conditions). The rigidity of leaflets 310a, 310b compared to the coupling regions 312a, 312b in this example allow them to prolapse (or collapse downstream) and allow blood flow with minimal restriction due to the exertion-related blood pressure. After the exertion-related blood pressure subsides, and the blood pressure returns to the normal blood pressure conditions or the elevated blood pressure conditions, then leaflets 310a, 310b can revert to the configurations shown in FIG. 3A or 3B, respectively.

The example bi-modal flow restrictor in FIGS. 4A-4C utilizes relatively elongated and relatively soft (or relatively more flexible) leaflets 410a, 410b coupled to frame 420 to restrict blood flow in response to elevated blood pressure (e.g., due to pulmonary overload). Leaflets 410a, 410b are coupled to frame 420 at coupling regions 412a, 412b. Leaflets 410a, 410b are relatively long, such that when they extend towards one another from the edge of the frame 420 they meet to close the open space between them when they are curved, as shown in FIG. 4B. In some examples, a length of leaflets 410a, 410b can be significantly longer (e.g., greater than 20% longer, or greater than 50% longer, or greater than 100% longer, or greater than 200% longer) than a radius 404 of the frame 420, wherein the radius is measured from a central axis 406 to the edge of frame 420 (or to coupling regions 412a, 412b). Leaflets 410a, 410b are relatively flexible compared to the coupling regions 412a, 412b. Therefore, when leaflets 410a, 410b move, due to a change in blood pressure, the leaflets 410a, 410b deform more than coupling regions 412a, 412b, as shown in FIGS. 4A-4C. For example, in FIG. 4C the leaflets have flexed to allow the leaflets to prolapse. In contrast, the coupling regions in the flow restrictor in FIG. 4C are in a relatively similar position as coupling regions 412a, 412b under normal blood pressure conditions in FIG. 4A.

FIG. 4A shows the bi-modal flow restrictor under normal blood pressure conditions (e.g., about 1 mmHg to about 15 mmHg), where leaflets 410a, 410b are spaced apart such that they are, for example, about 20% to about 80% closed (meaning that the open area between the inflow ends of leaflets 410a, 410b includes about 80% to about 20% of the total cross-sectional area of the lumen of the flow restrictor, under normal blood pressure conditions).

FIG. 4B shows the flow restrictor under elevated blood pressure conditions, where the blood pressure in blood vessel in FIG. 4B is higher than the blood pressure in the blood vessel 402 in FIG. 4A (e.g., from about 10 mmHg to about 25 mmHg, or from about 15 mmHg to about 25 mmHg). Leaflets 410a, 410b in FIG. 4B are spaced closer together (or touching) such that they are, for example, about 60% to about 100% closed (meaning that the open area between the inflow ends of leaflets 410a, 410b includes about 40% to about 0% of the total cross-sectional area of the lumen of the flow restrictor, under the elevated blood pressure conditions). Leaflets 410a, 410b closing in FIG. 4B reduces the amount of blood that can flow through the flow restrictor in this example, thereby lowering blood pressure downstream from leaflets 410a, 410b.

FIG. 4C shows the flow restrictor under exertion-related blood pressure conditions, where the blood pressure in blood vessel 402 in FIG. 4C is higher than the blood pressure in the blood vessel in FIGS. 4A and 4B (e.g., greater than about 25 mmHg, or greater than about 30 mmHg). Leaflets 410a, 410b in FIG. 4C are prolapsed (wherein leaflets 410a, 410b move in the downstream direction) such that they are, for example, about 5% to about 100% (or about 10% to about 90%) opened (meaning that the open area between the inflow ends of leaflets 410a, 410b includes about 5% to about 100% of the total cross-sectional area of the lumen of the flow restrictor, under the exertion-related blood pressure conditions). The flexibility of leaflets 410a, 410b in this example allows them to prolapse (or collapse downstream) and allow blood flow with minimal restriction due to the exertion-related blood pressure. After the exertion-related blood pressure subsides, and the blood pressure returns to the normal blood pressure conditions or the elevated blood pressure conditions, then leaflets 410a, 410b can revert to the configurations shown in FIG. 4A or 4B, respectively. In some examples, flow restrictors for blood vessels include bi-modal flow restriction assemblies.

In some examples, bi-modal flow restriction assemblies include a valve disposed within an outer docking frame. The leaflets of the valve move proportionally radially inward (closer to each other) in response to elevated venous pressure to restrict flow through a lumen of the valve, while the valve is sealed against a seat of the docking frame. Exertion-related higher blood pressures can form a gap between the valve and the outer docking frame through which blood is allowed to flow around the valve in this state. These bi-modal flow restrictors can provide a mechanically driven solution by which GFR can be improved in CKD patients, as an alternative (or in lieu of) conventional pharmaceutical treatments.

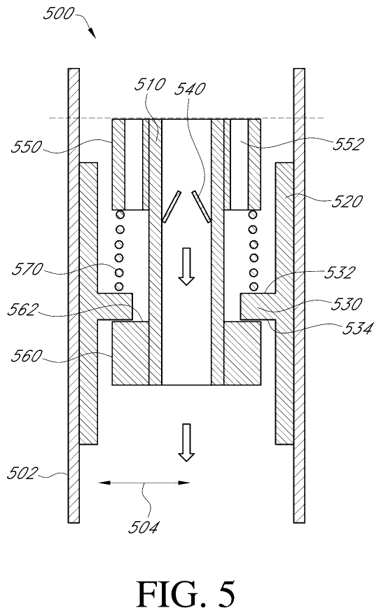

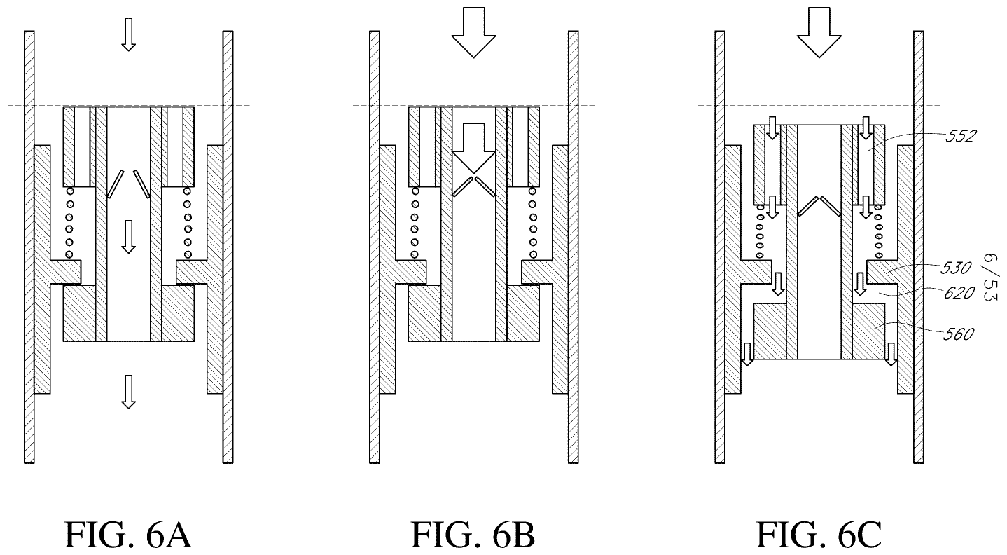

FIGS. 5 and 6A-6C show cross-sectional side view schematics of an example of a flow restrictor 500 with a bi-modal flow restriction assembly within a blood vessel 502. The bi-modal flow restriction assembly in this example includes a spring-biased inner valve 510 axially movable within an outer frame 520 (or outer docking frame). Outer frame 520 can be approximately cylindrical, in some examples. Outer frame 520 includes a narrower mid-portion designed to serve as a seat 530 having an inflow side 532 and an outflow end 534. Inner valve 510 includes a main lumen (or open space, through which blood can flow) with leaflets 540, an outwardly (i.e., in radial direction 504) extending inflow support portion 550 (or inflow ring) and an outwardly extending outflow support portion 560 (or outflow ring), disposed on opposite sides of seat 530. Optionally, the outwardly extending inflow support portion 550 can be in the form of an inflow ring surrounding the inflow portion of a frame of inner valve 510, and the outwardly extending outflow support portion 560 can be in the form of an outflow ring surrounding the inflow portion of the frame of inner valve 510. A compression spring 570 can be disposed between inflow side 532 of seat 530 and inflow support portion 550 (or inflow ring) of inner valve 510. Compression spring 570 is shown in FIGS. 5 and 6A-6C as a series of dots since the figures are cross-sections of the flow restrictors. Leaflets 540 can be similar to, and can function similar to, leaflets 110a, 110b in FIG. 1. In this example, leaflets 540 can optionally prolapse, but do not need to prolapse (e.g., as described in FIGS. 3C and/or 4C), because there is a separate mechanism to increase the blood flow through the flow restrictor in the case of exertion-related blood pressure. Flow restrictor can also optionally contain springs to bias leaflets 540 in some cases, for example as shown in FIG. 1.

FIG. 6A shows flow restrictor 500 under normal blood pressure conditions (e.g., from about 1 mmHg to about 15 mmHg), where leaflets 540 are spaced apart such that they are, for example, about 20% to about 80% closed (meaning that the open area between the inflow ends of leaflets 540 includes about 80% to about 20% of the total cross-sectional area of the lumen of the flow restrictor, under normal blood pressure conditions). Compression spring 570 is biasing inner valve 510 proximally in a free state to the configuration shown in FIG. 6A, such that a shoulder 562 of the outflow support portion 560 (or outflow ring) is pressed against outflow end 534 of outer frame seat 530, sealing inner valve 510 against seat 530 in a manner that allows blood flow only through the open area (or lumen) of inner valve 510, as shown in FIGS. 5 and 6A. In some cases, the seal between the inner valve 510 and the seat 530 of the outer frame 520 is not perfect and small amounts of blood can pass through the imperfect seal.

FIG. 6B shows the flow restrictor 500 under elevated blood pressure conditions (e.g., from about 10 mmHg to about 25 mmHg, or from about 15 mmHg to about 25 mmHg), where the blood pressure in the blood vessel in FIG. 6B is higher than the blood pressure in the blood vessel in FIG. 6A. In response to elevated pressure (e.g., due to pulmonary overload), the blood flow impinges against the leaflets (which can be spring-biased leaflets in some cases) causing them to move closer to each other to proportionally restrict (but not completely block) flow through the lumen of the inner valve, as shown in FIG. 6B.

FIG. 6C shows the flow restrictor under exertion-related blood pressure conditions (e.g., greater than about 25 mmHg, or greater than about 30 mmHg), where the blood pressure in the blood vessel in FIG. 4C is higher than the blood pressure in the blood vessel in FIGS. 4A and 4B, for example due to exercise or stress. The inflow support portion 550 (or inflow ring) can include one or more channels 552 through which blood can also flow, such that higher exertion-related elevated pressure may cause the blood flow to impinge against a shoulder 562 of outflow support portion 560 (e.g., ring) that is sufficient to move inner valve 510 distally while compressing compression spring 570, as shown in FIG. 6C. This distal movement distances outflow support portion 560 (e.g., ring) from seat 530, allowing blood flow through a gap 620 formed between the inner valve and the outer frame. This allows more blood to flow through bi-modal flow restrictor 500 at exertion-related blood pressures.

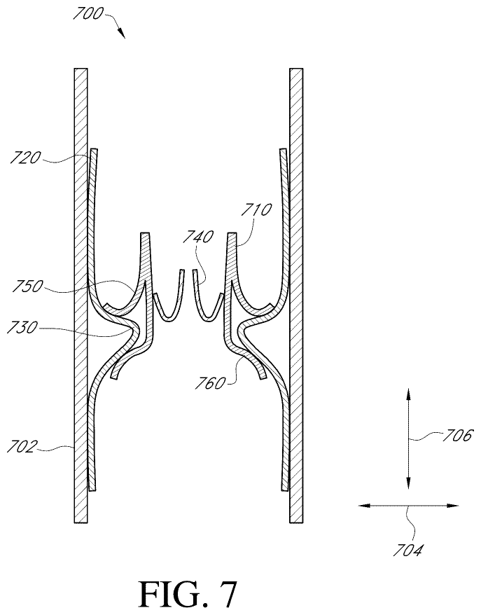

FIG. 7 shows an example of a cross-sectional side view schematic of a bi-modal flow restrictor 700 with a bi-modal flow restriction assembly within a blood vessel 702. Bi-modal flow restrictor 700 has similar components to, and operates similarly to, the bi-modal flow restrictor 500 in FIGS. 5-6C. Bi-modal flow restrictor 700 includes an inner valve 710 retained within an outer frame 720, where the inner valve 710 can move in an axial direction 706. The outflow support portion 760 is also inwardly deflectable or compressible (in a radial direction 704) in some cases.

The bi-modal flow restrictor 500 in FIGS. 5 and 6A-6C can be schematic representations of the working principle of the assembly described herein, instead of being implemented as an assembly. For example, spring 570 shown in FIGS. 5 and 6A-6C may not be implemented as a compression spring, but rather can be representative of a biased inflow support portion and/or outflow portion of the inner valve 710 that can transition between states, as described below. As such, bi-modal flow restrictor 700 in FIG. 7 is one possible implementation of the working principle of the assembly described with respect to FIGS. 5 and 6A-6C, however, other assemblies can be used to implement the same working principles.

Bi-modal flow restrictor 700 includes inner valve 710, which includes inflow support portion 750 and outflow support portion 760. Outflow support portion 760 can be provided as a portion of inner valve 710 that is flared radially outward in a free state thereof, in some examples. Leaflets 740 (similar to those described above, e.g., those in FIGS. 1-4C) are coupled to inner valve 710. Inner valve 710 is positioned within outer frame 720. Outer frame 720 includes seat 730. Inflow support portion 750 and outflow support portion 760 rest against opposing sides of seat 730 of outer frame 720.

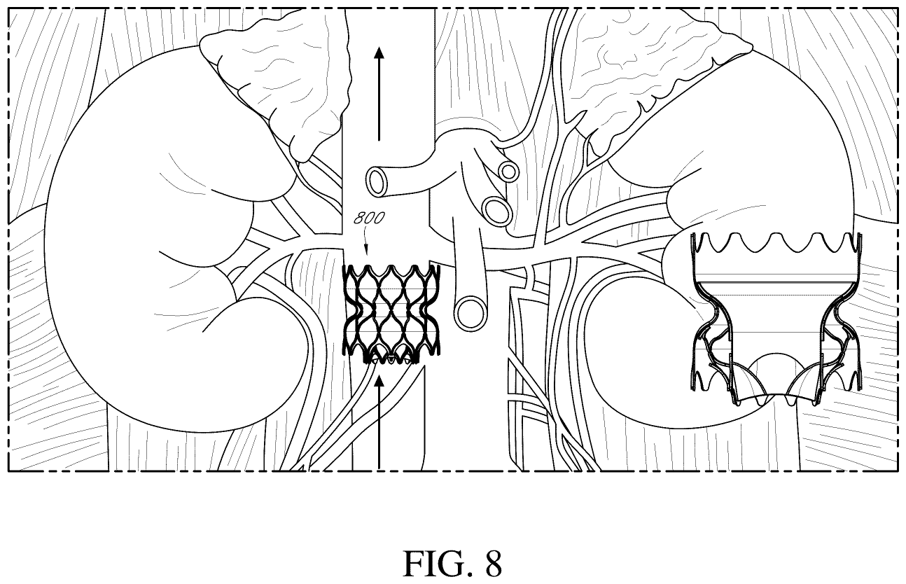

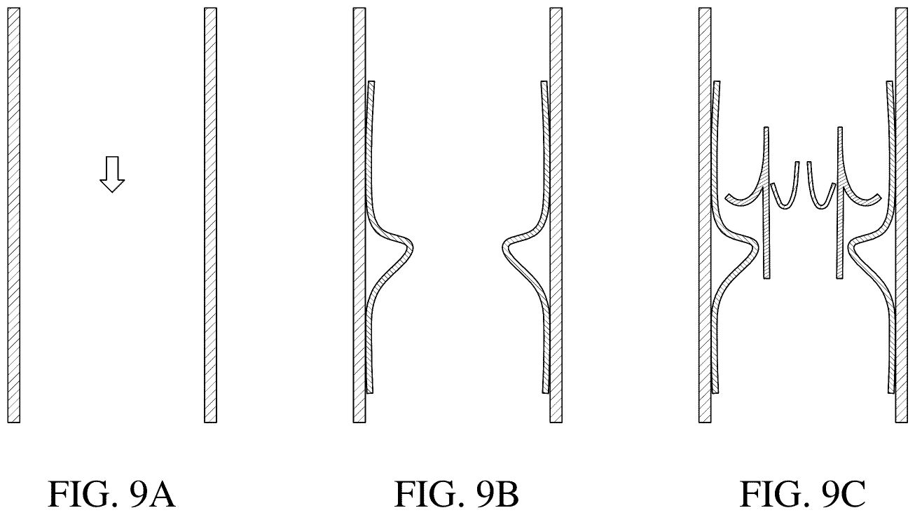

FIG. 8 shows an example position of a bi-modal flow restrictor 800 within the inferior vena cava (IVC) of a human patient. Bi-modal flow restrictor 800 includes the same components as bi-modal flow restrictor 700, and additionally bi-modal flow restrictor 800 includes a valve cover and a frame cover, which are described below.

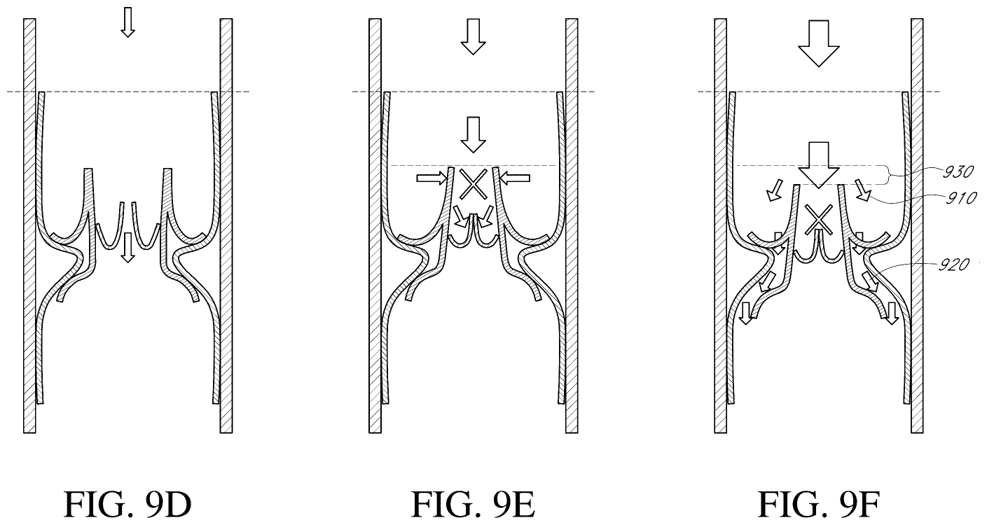

FIGS. 9A-9F show an example of stages of deployment and utilization of bi-modal flow restrictor 700. FIG. 9A shows a blood vessel (e.g., the IVC). Bi-modal flow restrictor 700 can be deployed by first deploying the outer frame in the blood vessel as shown in FIG. 9B, followed by gradual deployment and positioning of the inner valve therein.

The inner valve can be gradually deployed, for example, by pulling a capsule that first exposes the inflow support portion of the inner valve, as shown in FIG. 9C. After the inner valve is exposed, the inner valve is axially positioned to place the inflow support portion over the seat, which positions the outflow portion of the inner valve on an opposite end of the seat compared to the inflow support portion, as shown in FIG. 9C. The outflow portion of the inner valve is then pressed against the outflow end of the seat (to flare the outflow portion and form the outflow flared portion) in a manner that seals the inner valve against the outer frame, allowing blood flow only through the lumen of the inner valve, as shown in FIG. 9D. Once the outflow portion is flared, both the inflow support portion and the outflow flared portion are positioned on opposite ends of the seat, to prevent unintentional axial displacement of the inner valve in the axial direction.

FIG. 9D shows that inner valve (i.e., inner valve 710 in FIG. 7) is biased proximally in a free state to the configuration shown in FIG. 9D, such that outflow flared portion (i.e., outflow support portion 760 in FIG. 7) is pressed against an outflow end of seat (i.e., seat 730 in FIG. 7), which seals inner valve against seat in a manner that allows blood flow through the open area (or lumen) of inner valve (between leaflets (i.e., leaflets 740 in FIG. 7)). In some cases, the seal between outflow flared portion of the inner valve and the seat of the outer frame (i.e., outer frame 720 in FIG. 7) is not perfect and small amounts of blood can pass through the imperfect seal.

Bi-modal flow restrictor 700 shown in FIGS. 7 and 9A-9F can operate similarly to bi-modal flow restrictor 500 in FIG. 5. FIG. 9D shows the flow restrictor under normal blood pressure conditions (e.g., from about 1 mmHg to about 15 mmHg), FIG. 9E shows the flow restrictor under elevated blood pressure conditions (e.g., from about 10 mmHg to about 25 mmHg, or from about 15 mmHg to about 25 mmHg), and FIG. 4C shows the flow restrictor under exertion-related blood pressure conditions (e.g., greater than about 25 mmHg, or greater than about 30 mmHg). The leaflets respond to the changes in flow similarly to those of the flow restrictors described above, for example in FIGS. 4A-4C, wherein the leaflets close (thereby reducing the open area or lumen) in response to higher venous pressure. For example, in response to elevated pressure (e.g., due to pulmonary overload), the flow impinges against the leaflets (i.e., leaflets 740 in FIG. 7) causing them to move closer toward each other (in radial direction 704 in FIG. 7) to restrict (or proportionally restrict) flow through the lumen of the inner valve, as shown in FIG. 9E. The leaflets can restrict (or proportionally restrict) blood flow through the lumen of the inner valve, but leaflets do not necessarily completely block blood flow through the lumen of the inner valve. The “X” shown in FIGS. 9E and 9F about the leaflets indicate that the blood flow is restricted through the lumen, but not necessarily that the blood flow through the lumen is completely blocked.

FIG. 9F shows that exertion-related elevated blood pressure may cause the blood flow 910 around the inner valve (i.e., inner valve 710 in FIG. 7) to impinge against the outflow flared portion (i.e., outflow support portion 760 in FIG. 7) with sufficient pressure to move the inner valve distally by an amount 930 (e.g., by compressing or distorting the inflow support portions (i.e., inflow support portions 750 in FIG. 7). This distal movement of the inner valve distances the outflow flared portion from the seat (i.e., seat 730 in FIG. 7), allowing blood flow through a gap 920 formed between the inner valve and the outer frame at these higher exertion-related blood pressures, as shown in FIG. 9F.

In some examples, the flared outflow portion of the inner valve can be inwardly deflectable or compressible (e.g., made of a shape-memory material such as Nitinol), such that exertion-related elevated blood pressure can cause the blood flow to impinge against the flared outflow portion, with sufficient force to deflect it radially inward (in radial direction 704 in FIG. 7). In such cases, gap 920 between the inner valve and the surrounding outer frame, through which blood can flow at these higher exertion-related pressures, can be caused by the inner valve moving axially and/or by the outflow flared portion deflecting radially inwards.

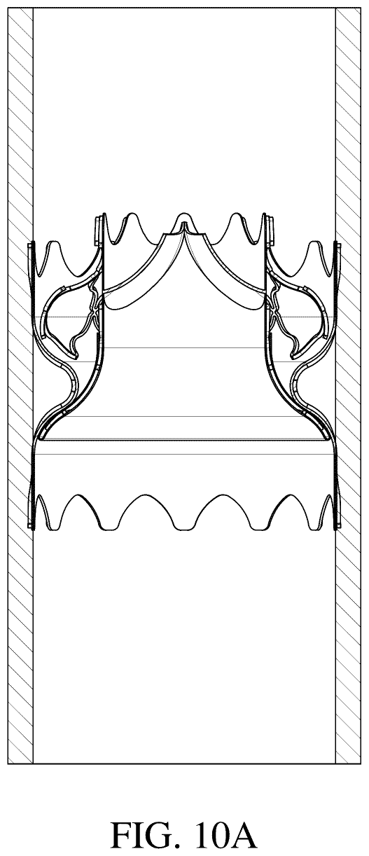

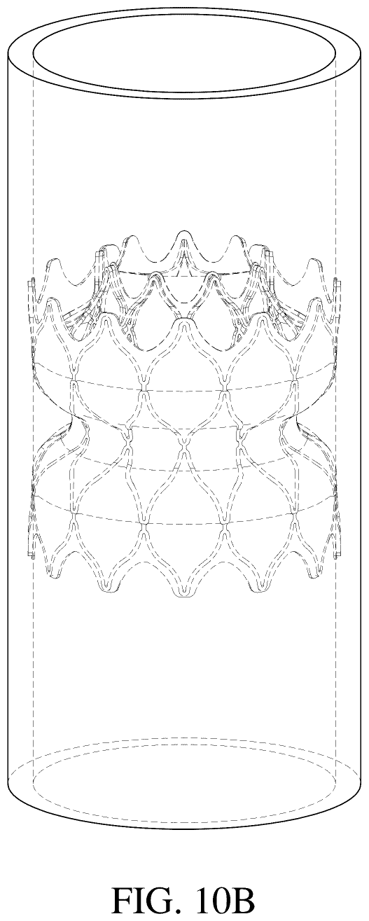

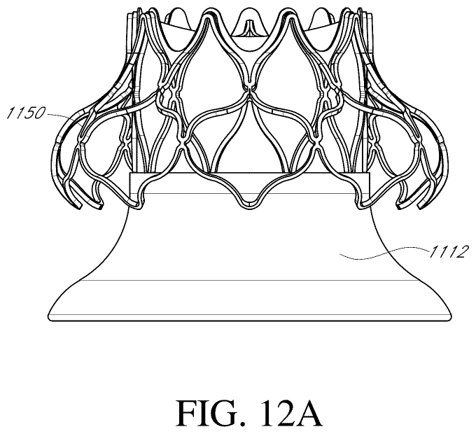

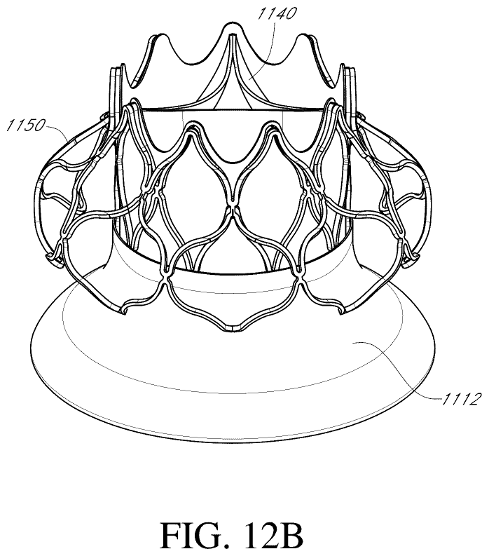

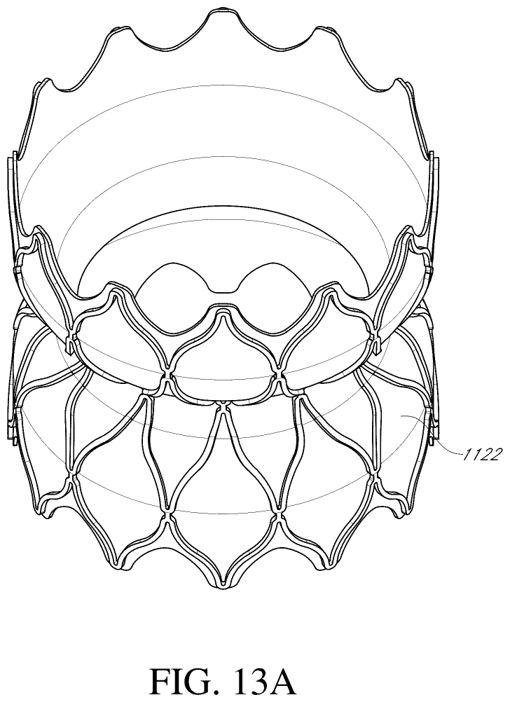

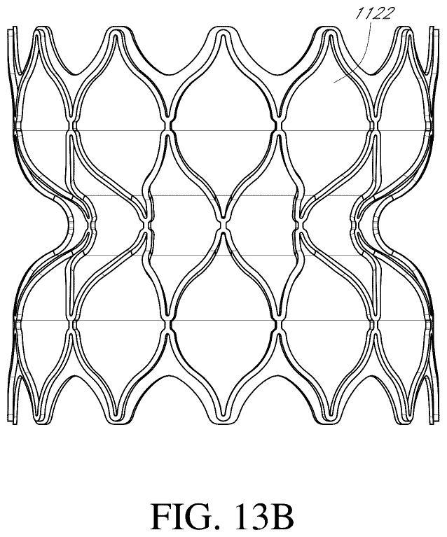

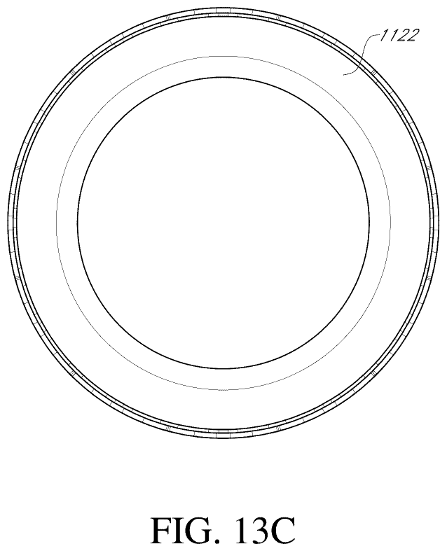

FIGS. 10A-14B show examples of proposed bi-modal flow restrictor assemblies (or components) implementing the working principle of the assembly described with respect to FIGS. 5 and 6A-6C. Some views of the bi-modal flow restrictor assemblies in FIGS. 10A-14B show the inner valve (e.g., in FIGS. 12A-12D) and/or the outer frame (e.g., in FIGS. 13A-13C) further including covers to block flow around the inner valve when the flared portion is pressed against the docking frame, and to direct blood flow through a gap between the inner valve and the outer frame under elevated exertion-related pressures. The outflow flared portion of the inner valve in FIGS. 10A-14B is covered by the inner valve cover 1112.

FIGS. 10A-10B show a cut-away side view and an isometric view, respectively of an example of a bi-modal flow restrictor similar to that shown in FIGS. 7-9F.

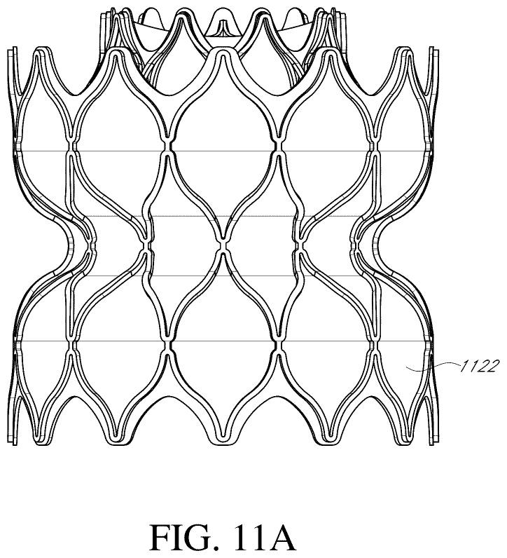

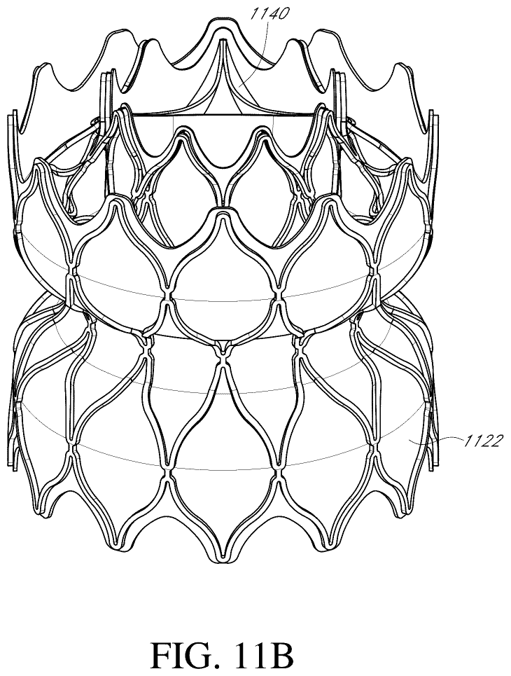

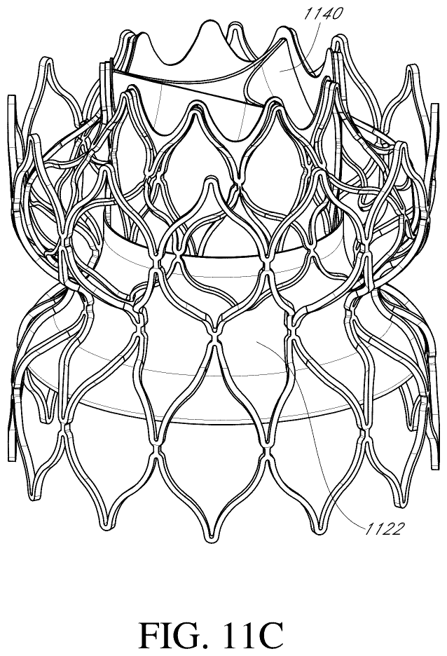

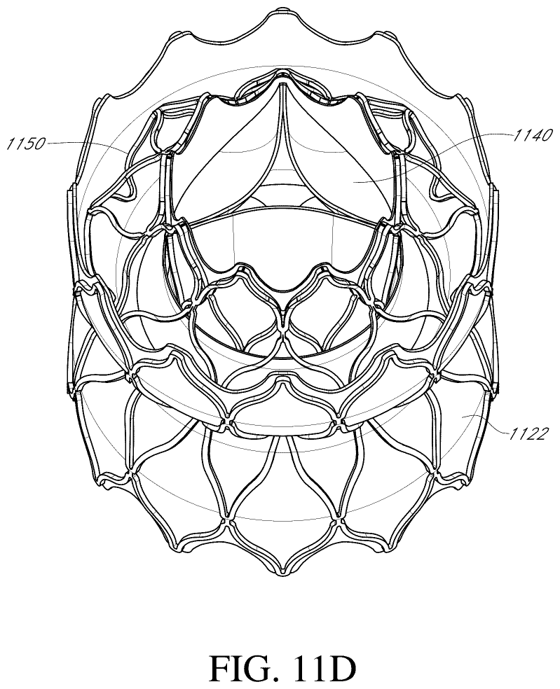

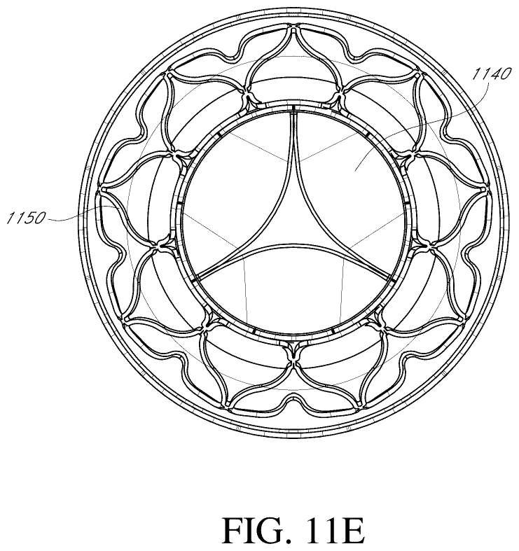

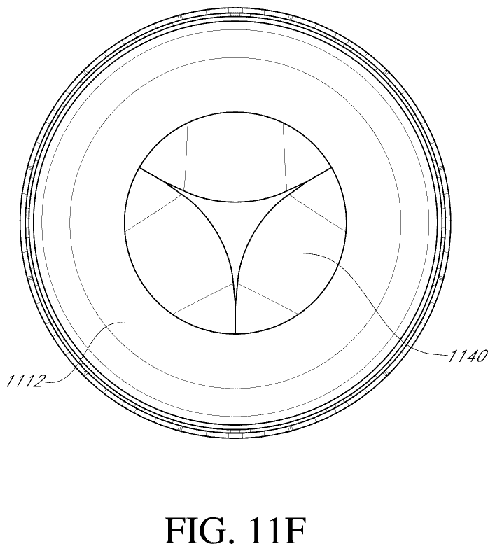

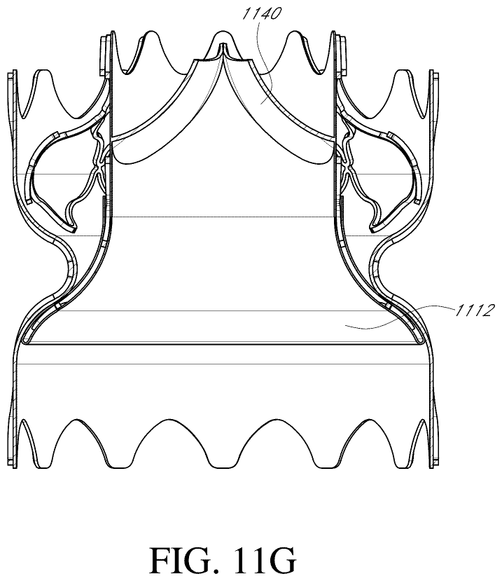

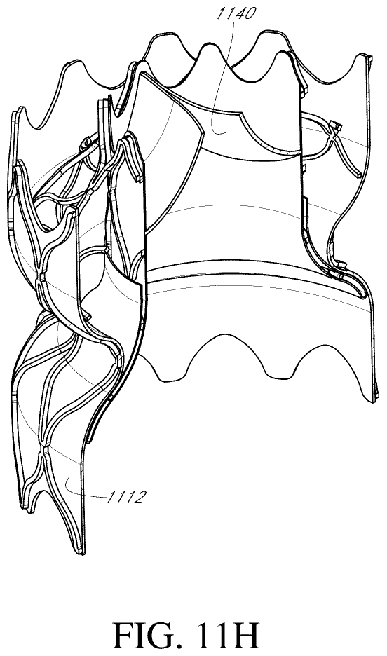

FIGS. 11A-11H show isometric and cut-away views, from different angles and with different components removed to show the assemblies, of an example of a bi-modal flow restrictor similar to that shown in FIGS. 7-9F, including an inner valve cover 1112, an outer frame cover 1122, an inflow support portion 1150, and leaflets 1140. FIG. 11E shows a view from the top where the inflow support portion 1150 is visible, and FIG. 11F shows a view from the bottom where the outflow flared portion covered by inner valve cover 1112 is visible.

FIGS. 12A and 12B show isometric views from different angles of an inner valve of an example of a bi-modal flow restrictor similar to that shown in FIGS. 7-9F, including an inner valve cover 1112, an inflow support portion 1150, and leaflets 1140.

FIGS. 13A-13C show isometric views from different angles of an outer frame of an example of a bi-modal flow restrictor similar to that shown in FIGS. 7-9F, including outer frame cover 1122.

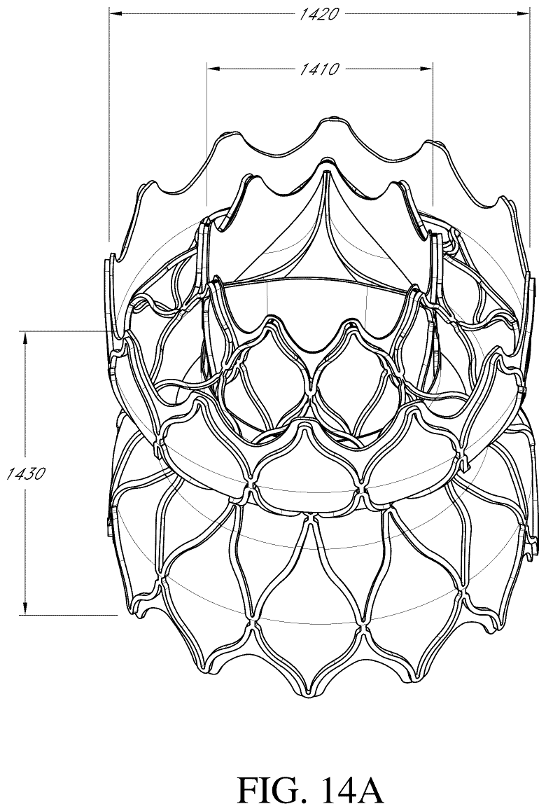

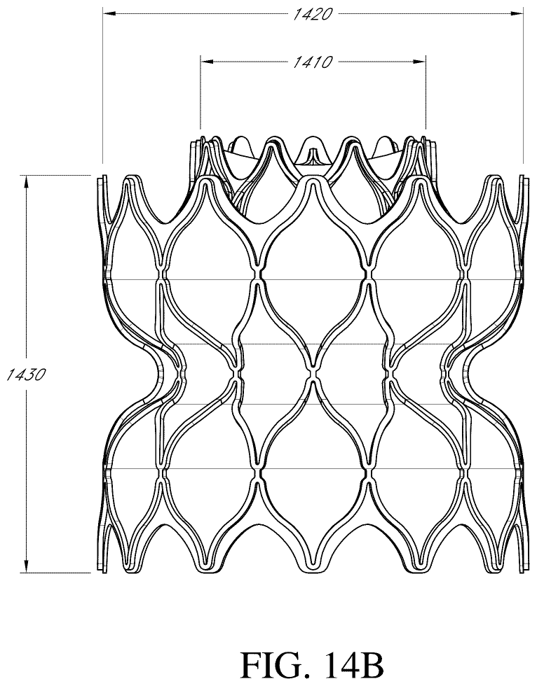

FIGS. 14A-14B show isometric views from different angles of an example of a bi-modal flow restrictor similar to that shown in FIGS. 7-9F, including some example dimensions. In this example, a diameter 1410 of the inner valve is about 13 mm, a largest diameter 1420 of the outer frame is about 24 mm, and the length 1430 of the bi-modal flow restrictor is about 23 mm. In other examples, the diameter 1410 of the inner valve can be from about 2 mm to about 20 mm, or from about 10 mm to about 15 mm. In other examples, the diameter 1420 of the outer frame can be from about 5 mm to about 40 mm, or from about 20 mm to about 30 mm. In other examples, the diameter 1420 of the bi-modal flow restrictor can be from about 5 mm to about 40 mm, or from about 20 mm to about 30 mm. An average diameter of blood vessels in which the present flow restrictors can be positioned can vary, for example, from about 5 mm to about 30 mm, and the flow restrictors described herein (e.g., in FIGS. 1-14B) can be different sizes to accommodate differently sized blood vessels. For example, the diameter of the inner valve can be from about 2 mm to about 25 mm, a largest diameter of the outer frame can be from about 5 mm to about 35 mm, and the length of the bi-modal flow valve can be from about 15 mm to about 45 mm. In some cases, the bi-modal flow restrictor (e.g., the outer frame) may be oversized, and the dimensions described herein are dimensions (or target dimensions) of the device in situ. In some cases, manufactured dimensions of the (bi-modal) flow restrictors described herein may be larger or smaller than what is described herein.

In some examples, flow restrictors for blood vessels include flaps movable by control elements (e.g., flexible wires) extending through helical tubes.

In some examples, flow restrictors that include flaps attached to and pivotable about the outflow end of a stent. The flaps are coupled to and pullable by control elements (e.g., wires) that extend through expandable helical tubes. Tube expansion in response to elevated pressure and/or increased blood volume can shorten the exposed portion of the wires, so as to pull the flaps radially inward (closer to each other). These flow restrictors can provide a mechanically driven solution by which GFR can be improved in CKD patients, as an alternative (or in lieu of) conventional pharmaceutical treatments.

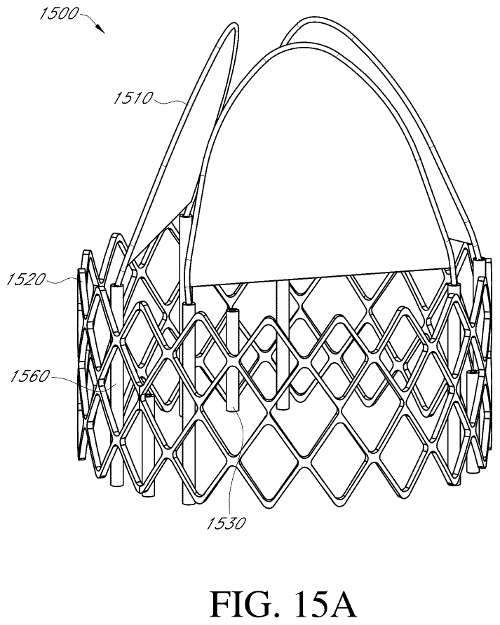

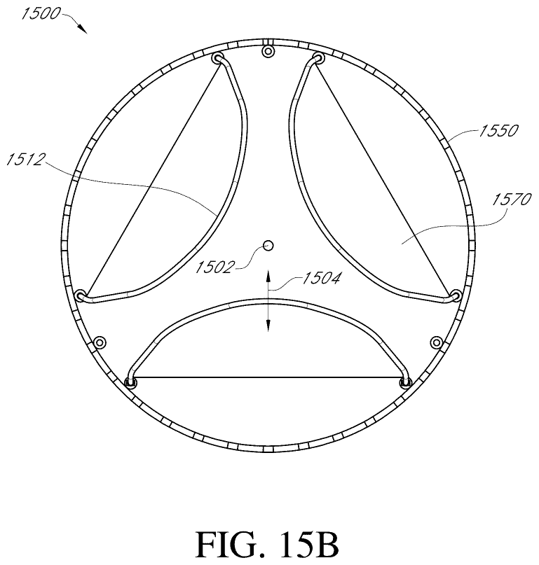

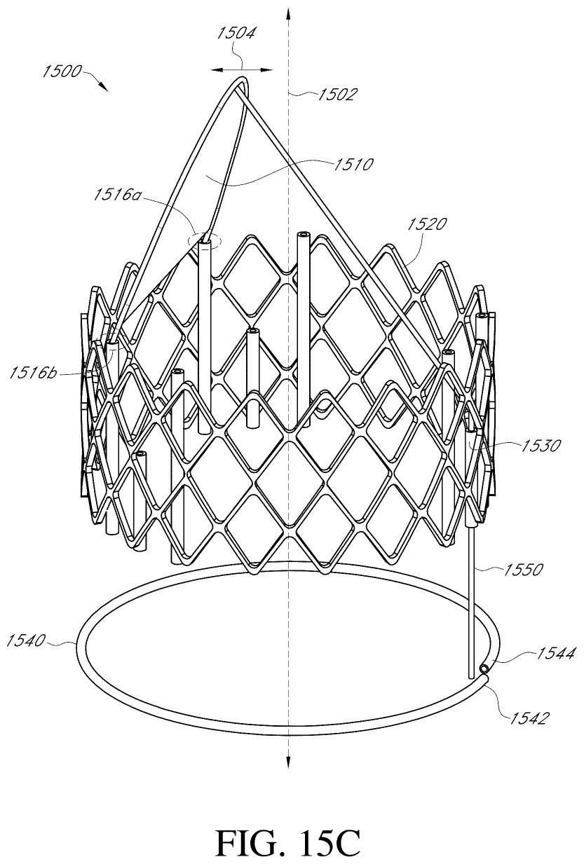

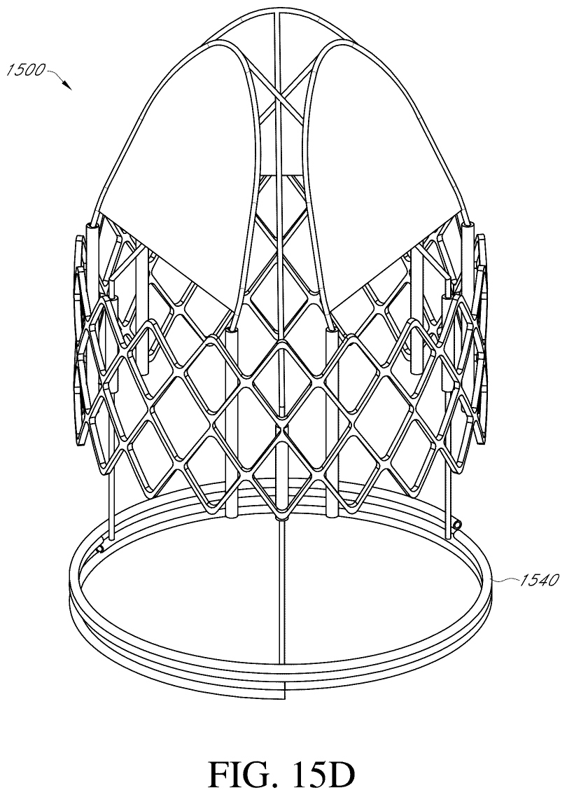

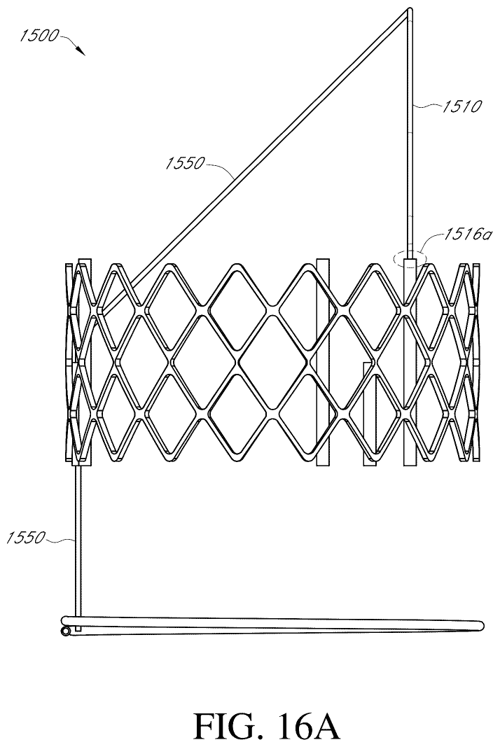

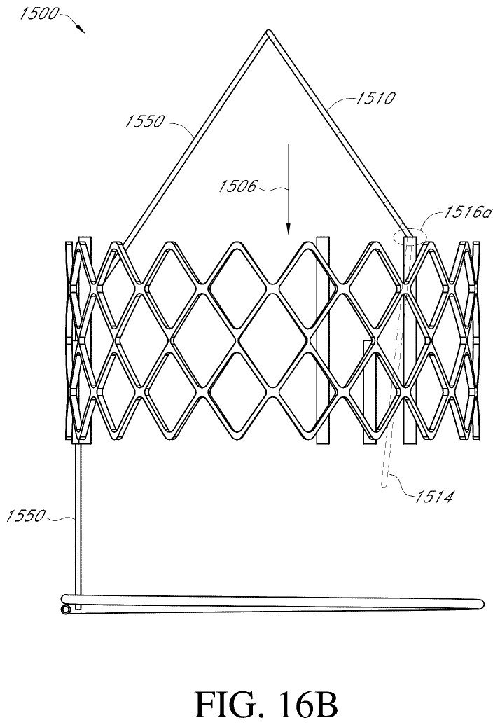

FIGS. 15A-15D show isometric views and a top-down view (in FIG. 15B) of an example of a flow restrictor 1500 that can be positioned within a blood vessel. The blood flow direction is up (i.e., in the opposite direction of 1506 in FIG. 16B) in the examples shown in FIGS. 15A-15D and 16A-16B. Flow restrictor 1500 includes one or more flaps 1510 with inflow ends coupled to a frame 1520 using support tubes 1560, such that an outflow end 1512 (or flap edge, or resilient edge) of the flap 1510 is movable in a radial direction 1504 approximately perpendicular to a central axis 1502 of frame 1520. Each flap 1510 is attached to, and movable by, a control element 1550 (e.g., a flexible tension member, a flexible wire, a suture, a string, a cable, or the like) extending through a guide tube 1530, and through a helical tube 1540. The term “helical” as used herein, refers to a shape of a curve that lies on the curved surface of a cylinder, or on the surface of a prism with a non-circular base, for example on a prism with an approximately ovular base or other shaped base (e.g., rectangular or irregular).

FIGS. 15A-15B show a part of flow restrictor 1500, with the helical tubes and flexible tension members removed from view for clarity. Frame 1520 can be an expandable frame or a stent, which can be covered or uncovered. The one or more flaps 1510 can extend from the outflow end of frame 1520, for example.

Flaps 1510 can include a resilient edge, for example, that can be formed from Nitinol or other suitable pre-shaped materials that can extend through and/or from the upper ends of support tubes 1560 (or posts) attached to frame 1520. Flexible cover 1570 can extend over flaps 1510, and optionally can accommodate changes in shape of the optionally expandable frame 1520. The flexible cover 1570 can be made of polyurethane (PU), other fabrics or suitable synthetic materials, or tissue (e.g., pericardium). Flaps 1510 can be coupled to frame 1520 such that they can flex (toward or away from each other) around the upper ends of the support tubes to which they are coupled. For example, flaps 1510 can be coupled to support tubes 1560 using pivotable couplings 1516a, 1516b that enable the flaps 1510 to pivot (or flex) around the upper ends of the support tubes 1560 to which they are coupled.

The flow restrictor 1500 in FIGS. 15C-15D further includes helical tubes. FIG. 15C shows an example with a flap 1510 coupled to a control element 1550. FIG. 15D shows an example with three flaps, each coupled to a control element 1550. Each control element 1550 is anchored to a proximal end 1542 of helical tube 1540, extends along a channel inside helical tube 1540, and exits out of a distal end 1544 of helical tube 1540. After exiting out of a distal end 1544 (e.g., opening), control element 1550 extends through guide tube 1530 (which is coupled to frame 1520). Guide tubes 1530 can be optionally shorter than the support tubes 1560, in some cases. Control element 1550 then extends out of an outflow end of guide tube 1530 and is coupled to an outflow end 1512 (or upper end, or flap edge, or resilient edge) of a corresponding flap 1510 at the opposite side of frame 1520.

FIGS. 16A and 16B show an example of flow restrictor with one flap 1510 in a relatively extended and in a relatively retracted position, respectively. The one or more helical tubes 1540 are coupled to the blood vessel (e.g., to an inner surface of the blood vessel) in such a way that the helical tubes 1540 may expand (i.e., the length of the internal channel between proximal end 1542 and distal end 1544 may become longer) in response to elevated pressure (e.g., due to pulmonary overload) and/or enlargement of the blood vessel (e.g., due to increase in blood volume). For example, a length of the channel within a helical tube 1540 can expand by about 1 mm to about 2 mm in response to elevated pressure and/or enlargement of the blood vessel. The expansion of a helical tube 1540 may cause the control element 1550 to pull the flaps radially inward, as shown in FIG. 16B. The flaps 1510 moving radially inward (or retracting), as shown in FIG. 16B, causes a greater portion of the cross-sectional area of flow restrictor 1500 to be obstructed, thereby serving to restrict blood flow through flow restrictor 1500 in response to an increase in blood pressure.

In some examples, helical tubes 1540 are coupled to the blood vessel such that when the blood vessel expands or changes cross-sectional size or shape (e.g., due to an increase in blood pressure or blood volume), the length of the internal channel between proximal end 1542 and distal end 1544 of helical tube 1540 may change (e.g., become shorter or become longer). In some examples, helical tubes 1540 can be coupled to the blood vessel at a plurality of points such that when the blood vessel expands or changes cross-sectional size or shape (e.g., due to an increase in blood pressure), then forces can act on helical tube 1540 causing it to take on a similar shape as the cross-sectional shape of the blood vessel. In some examples, helical tube 1540 is made from an elastic material, or a material with a modulus of elasticity such that forces from the blood vessel (e.g., caused by an increase in blood pressure), are sufficient to cause the length of the internal channel of helical tube 1540t to change. In some cases, helical tube 1540 is naturally larger than the blood vessel and helical tube 1540 is compressed upon insertion into the blood vessel. In some cases, the linkages of helical tubes 1540 and control element 1550 are fixed, such that a change in diameter (or effective diameter) of the blood vessel results in a change in occlusion (based on the fixed length of the control element 1550). In some cases, control elements 1550 are relatively rigid.

Flow restrictor 1500 can be positioned (or implanted) within a blood vessel, such as the IVC and/or in a vein upstream from the renal veins. When flow restrictor 1500 is implanted in a blood vessel (e.g., the IVC upstream from the renal veins), elevated venous pressure (or an enlargement of the blood vessel) may expand the helical tubes, thereby pulling on the flaps in a manner that narrows the open area through which blood can flow. This may have the effect of lowering blood pressure downstream from the flaps, such as at the renal veins, which can eventually increase the renal pressure gradient and improve kidney filtration rates. When the blood pressure is lowered, helical tubes 1540 may recompress, releasing a longer portion of control element 1550, and allowing flaps 1510 to reassume their upwardly biased open state, as shown in FIG. 16A.

In some examples, flow restrictor 1500 can be a bi-modal flow restrictor that is designed to allow the flaps to prolapse further downward (in the direction 1506) when the venous pressure exceeds an upper threshold (e.g., greater than about 25 mmHg, or greater than about 30 mmHg) and/or when the blood vessel enlarges beyond a first threshold. Such blood pressures may be indicative of exertion-related blood pressures (e.g., due to activity or stress), and the flap(s) 1510 prolapsing allow unrestricted (or minimally restricted) blood flow through flow restrictor 1500. For example, the flap could prolapse into position 1514 in response to the blood pressure exceeding the upper threshold. After the blood pressure decreases, then flap 1510 can be configured to return to a position, such as those shown in FIGS. 16A and 16B, for example using a spring or due to flap(s) 1510 being naturally biased in the position shown in FIG. 16A.

In some examples, flaps 1510 of flow restrictor 1500 can be actively controllably moved via external activation. Any of the active mechanisms used to actively move (or actuate) a component of a flow restrictor describe herein (e.g., using Wi-Fi, electrical, magnetic, or heating mechanisms, or the like) can be used to move flaps 1510 in flow restrictor 1500.

In some examples, flow restrictors for blood vessels include flaps movable by push or pull members.

In some examples, bi-modal flow restrictors include flaps attached to and pivotable about the outflow end of a frame (or stent) with an oval-shaped cross-section. The frame is configured to assume a less oval-shaped profile in response to elevated blood pressure, so as to increase its cross-sectional area, while the flaps are connected to push rod or pull wires configured to transition the flaps between a tilted state (relative to the plane of the outflow end) in low pressures, and to push or pull the flaps toward alignment with the outflow plane in response to elevated pressures, thereby restricting blood flow. These bi-modal flow restrictors can provide a mechanically driven solution by which GFR can be improved in CKD patients, as an alternative (or in lieu of) conventional pharmaceutical treatments.

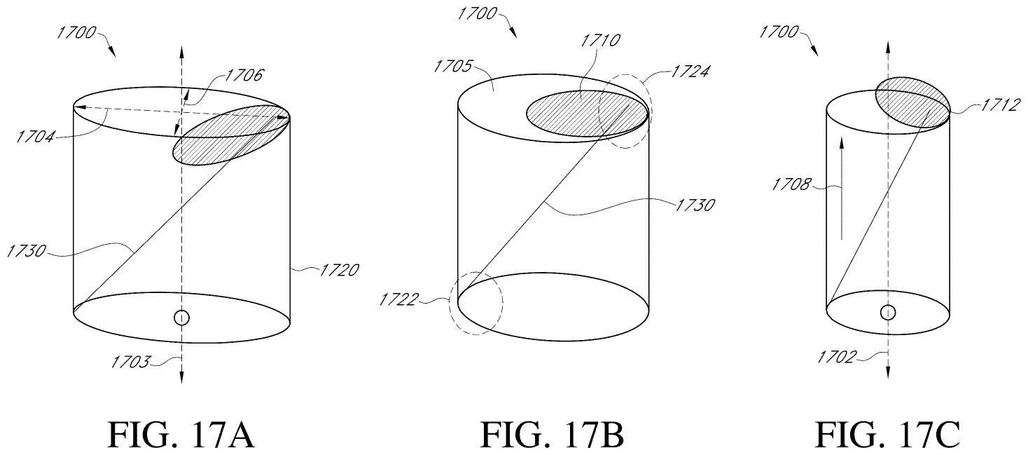

FIGS. 17A-28A show isometric and top-down views of flow restrictors that include one or more flaps attached to, and movable by, rigid push members or (flexible or rigid) pull members.

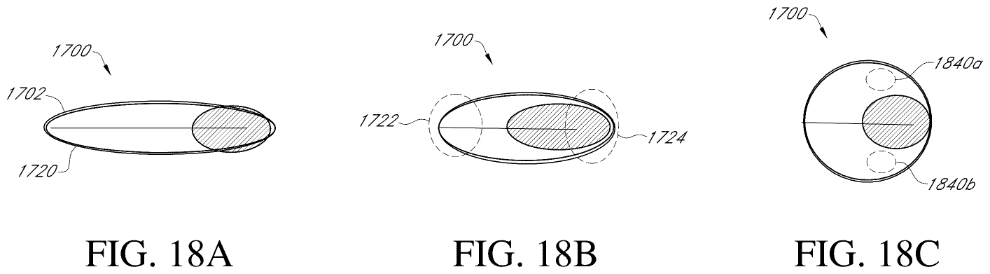

FIGS. 17A-17C show isometric views of an example flow restrictor 1700 that includes one or more flaps 1710 attached to, and movable by, rigid push member 1730. FIGS. 18A-18C show top-down views of flow restrictor 1700, in states that correspond to those shown in FIGS. 17A-17C, respectively. Flow restrictor 1700 is shown in FIGS. 17A and 18A under normal blood pressure conditions (e.g., from about 1 mmHg to about 15 mmHg), in FIGS. 17B and 18B under elevated blood pressure conditions (e.g., from about 10 mmHg to about 25 mmHg, or from about 15 mmHg to about 25 mmHg), and in FIGS. 17C and 18C under exertion-related blood pressure conditions (e.g., greater than about 25 mmHg, or greater than about 30 mmHg). Flow restrictor 1700 includes an oval frame 1720 (or stent) with a flap 1710 hinged to the outflow end of oval frame 1720. Flap 1710 is moveably coupled (hinged) to the outflow end of oval frame 1720 at position 1712. In practice, flap 1710 may not be coupled to frame 1720 at a single point, but rather over a region around position 1712, such as in region 1724. The direction 1708 of blood flow through the flow restrictor is also shown. In some cases, flap 1710 is also oval shaped, as shown in FIGS. 17A-18C. However, in other cases, flap 1710 can be shapes other than ovals, such as circles, rectangles, or irregular shapes. In some cases, two or more flaps similar to flap 1710 can be used to further restrict the lumen through frame 1720.

FIGS. 18A-18C show both the frame 1720 and a blood vessel 1702 surrounding frame 1720. However, a cross-section of the frame 1720 (approximately perpendicular to a central axis 1703 of frame 1720) is significantly oversized relative to a cross-section of blood vessel 1702, and therefore, blood vessel 1702 is in contact with frame 1720 in all states shown in FIGS. 18A-18C.

Flap 1710 is coupled to frame 1720 in a region 1724 at the outflow end of frame 1720, a first end of a relatively rigid push member 1730 (such as a rod or rigid wire) is coupled to flap 1710, and the second end of relatively rigid push member 1730 is coupled to an opposite side of frame 1720 in a region 1722 at the inflow end of the frame. The axis 1704 of the oval cross-section intersects frame 1720 in region 1722, which can be referred to as a long-axis region of frame 1720. The axis 1704 of the oval cross-section also intersects frame 1720 in region 1724 at the opposite side of the frame, which can also be referred to as a long-axis region of frame 1720. Note that flap 1710 is coupled to frame 1720 in region 1724, and therefore, rigid push member 1730 cannot be coupled to flap 1710 at a point where the flap 1710 is coupled to the frame 1720 (since in such a case the rigid push member 1730 would not be able to move the flap 1710). In this example, rigid push member 1730 is coupled to flap 1710 at a point that is a spaced away from a point where the flap 1710 is coupled to the frame 1720, in a configuration that enables rigid push member 1730 to move flap 1710 when oval-shaped frame 1720 is distorted (thereby becoming more cylindrical) as shown in FIGS. 17A-18C.

FIGS. 17A and 18A show flow restrictor 1700 under normal blood pressure conditions (e.g., from about 1 mmHg to about 15 mmHg). Frame 1720 is configured to be significantly oversized compared to the blood vessel, and to assume an oval shape that forces a wall of the blood vessel to be similarly shaped at these normal (relatively low) blood pressures. Since frame 1720 is oversized compared to the blood vessel 1702, the frame 1720 may be in contact with the blood vessel 1702 in all states shown in FIGS. 17A-18C. In the initial state shown in FIGS. 17A and 18A, the length of the rigid push member 1730 (or push rod), the size of frame 1720, the coupling region 1722 of rigid push member 1730 to frame 1720, the coupling location of rigid push member 1730 to flap 1710, and the coupling region 1724 of the flap 1710 to the frame 1720, are configured to tilt the flap downward (opposite the direction 1708 of the blood flow).

FIGS. 17B and 18B show flow restrictor 1700 under elevated blood pressure conditions (e.g., from about 10 mmHg to about 25 mmHg, or from about 15 mmHg to about 25 mmHg). When the device is implanted in a blood vessel (e.g., the IVC, for example upstream from the renal veins), elevated venous pressure may force the frame 1720 and blood vessel 1702 to assume a less oval shape, as shown in FIGS. 17B and 18B, which may cause the rigid push member 1730 to push against the flap 1710, causing the flap 1710 to move upwards (i.e., in the direction 1708 of the blood flow) and restrict a greater portion of the outflow, thereby restricting blood flow at higher pressures. In other words, when blood vessel 1702 changes shape (e.g., due to an increase in blood pressure), then frame 1720 may also change shape (due to the forces on the frame 1720 inside blood vessel 1702). Then when the frame 1720 changes shape to assume a less oval shape, then axis 1704 may become shorter, and rigid push member 1730 may push against the flap 1710 moving it up (in direction 1708). In this example, the flap 1710 is configured to transition between a tilted state (relative to a plane 1705 of the outflow end of frame 1720, wherein axis 1704 and axis 1706 are on the plane 1705) in low (or normal) pressure ranges, and to move flap 1710 toward alignment with the outflow plane of frame 1720 in response to elevated pressures, thereby restricting blood flow.

FIGS. 17C and 18C show flow restrictor 1700 under exertion-related blood pressure conditions (e.g., greater than about 25 mmHg, or greater than about 30 mmHg). The frame 1720 can be designed in this example to be significantly oversized relative to the blood vessel 1702, such that when the venous pressure exceeds an upper threshold (e.g., greater than about 25 mmHg, or greater than about 30 mmHg), which may be indicative of the pressures during exercise activity or stress, the frame 1720 and blood vessel 1702 therearound assume a circular (or nearly circular, or more circular) cross-sectional profile, which has an increased cross-sectional area relative to the oval profile. The exertion-related blood pressure can thereby cause the rigid push member 1730 to further push the flap 1710 upwards (i.e., in the direction 1708 of the blood flow), as shown in FIGS. 17C and 18C, such that the projected area of the flap 1710 restricts a smaller portion of the outflow, thereby allowing more (or less restricted) blood flow therethrough. Additionally, in some cases, the more circular cross-section of frame 1720 and blood vessel 1702 may allow blood to flow around the sides of flap 1710, for example through regions 1840a, 1840b, because flap 1710 is approximately oval shaped. When the blood pressure is lowered, the frame 1720 may reassume the more oval shape and the rod may pull the flap back downwards, shown in FIGS. 17A-17B and 18A-18B.

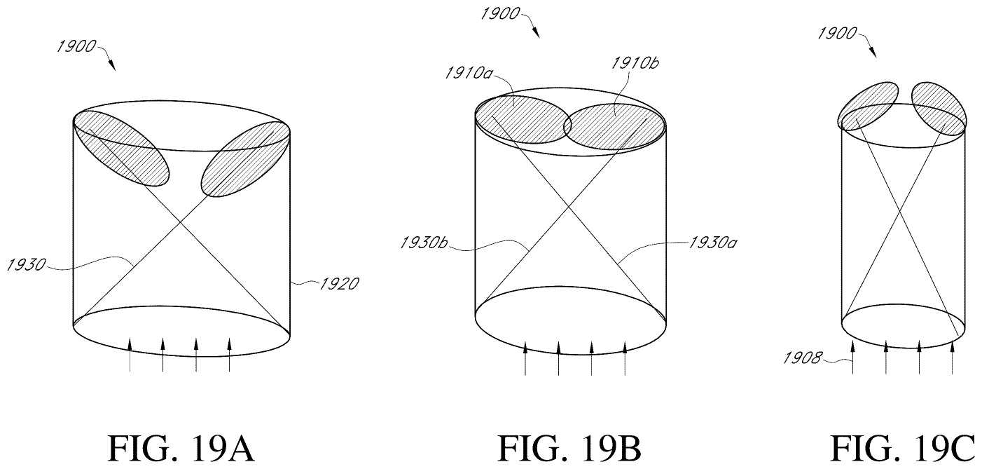

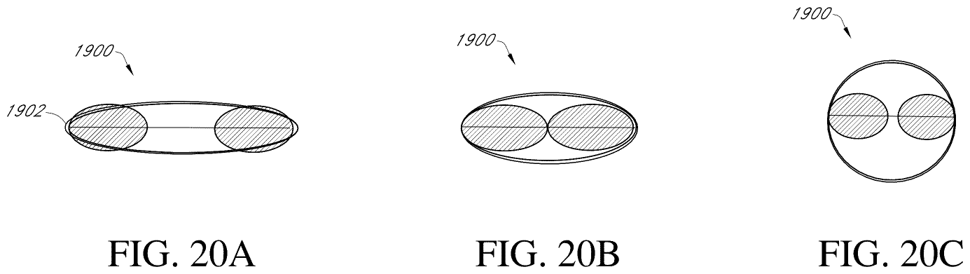

FIGS. 19A-20C show isometric and top-down views of an example flow restrictor 1900, which is similar to flow restrictor 1700 shown in FIGS. 17A-18C, but flow restrictor 1900 has two flaps 1910a, 1910b attached to opposite sides of frame 1920 instead of one flap. FIGS. 20A-20C show top-down views of flow restrictor 1700, in states that correspond to those shown in FIGS. 19A-19C, respectively. Flow restrictor 1900 is shown in FIGS. 19A and 20A under normal blood pressure conditions (e.g., from about 1 mmHg to about 15 mmHg), in FIGS. 19B and 20B under elevated blood pressure conditions (e.g., from about 10 mmHg to about 25 mmHg, or from about 15 mmHg to about 25 mmHg, or from about 10 mmHg to about 30 mmHg), and in FIGS. 19C and 20C under exertion-related blood pressure conditions (e.g., greater than about 25 mmHg, or greater than about 30 mmHg). In some cases, the flaps 1910a, 1910b can each be smaller than flap 1710 in FIGS. 17A-18C. In some cases, flaps 1910a, 1910b are oval shaped, as shown in FIGS. 19A-20C. However, in other cases, flaps 1910a, 1910b can be shapes other than ovals, such as circles, rectangles, or irregular shapes. The components and operation of flow restrictor 1900 are similar to or the same as those of flow restrictor 1700 in FIGS. 17A-18C described above, except that the two flaps 1910a, 1910b of flow restrictor 1900 are actuated by two rigid push members 1930a, 1930b, instead of only one flap 1710 being actuated in flow restrictor 1700. Direction 1908 shows the direction of blood flow through blood vessel 1902. In some cases, one larger flap (similar to flap 1710 in FIGS. 17A-18C) can be used to further restrict the lumen through frame 1720.

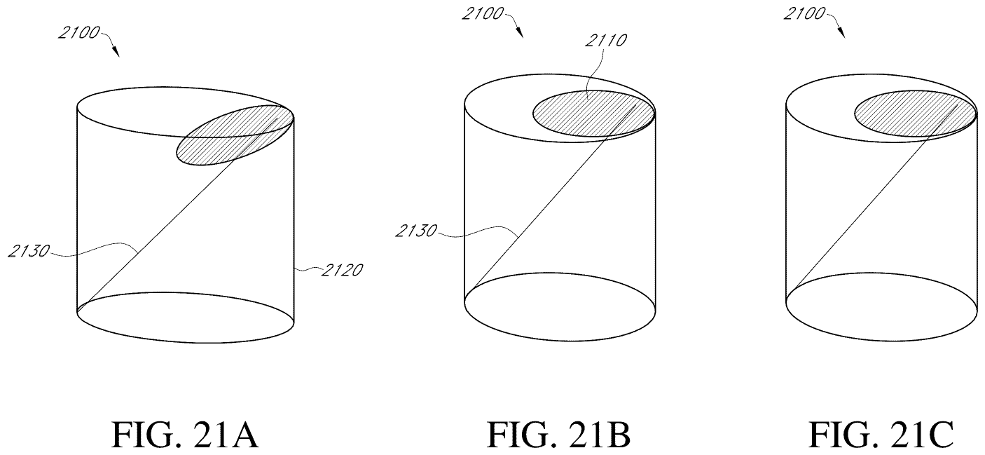

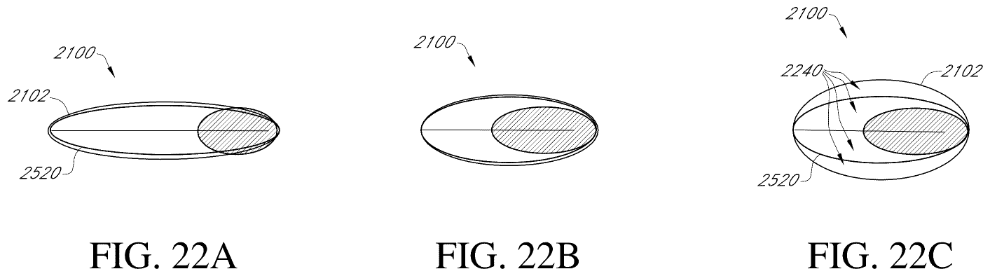

FIGS. 21A-22C show isometric and top-down views of an example flow restrictor 2100, which is similar to flow restrictor 1700 shown in FIGS. 17A-18C, however, flow restrictor 2100 has a frame 2120 that is not oversized relative to blood vessel 2102, and frame 2120 can expand from an oval shape to a less oval shape, but not necessarily to a circular (or nearly circular) profile. FIGS. 22A-22C show top-down views of flow restrictor 1700, in states that correspond to those shown in FIGS. 21A-21C, respectively. Flow restrictor 2100 is shown in FIGS. 21A and 22A under normal blood pressure conditions (e.g., about 1 mmHg to about 15 mmHg), in FIGS. 21B and 22B under elevated blood pressure conditions (e.g., from about 10 mmHg to about 25 mmHg, or from about 15 mmHg to about 25 mmHg, or from about 10 mmHg to about 30 mmHg), and in FIGS. 21C and 22C under exertion-related blood pressure conditions (e.g., greater than about 25 mmHg, or greater than about 30 mmHg).

The components and operation of flow restrictor 2100 in FIGS. 21A-22C are similar to or the same as those of flow restrictor 1700 in FIGS. 17A-18C described above, except that frame 2120 is not oversized (or is minimally oversized) relative to blood vessel 2102. In the example shown in FIGS. 21A-22C, blood vessel 2102 can tightly surround the frame 2120 and be forced to assume a similar profile at the low (or normal) blood pressure conditions shown in FIGS. 21A and 22A and at the elevated blood pressure conditions shown in FIGS. 21B and 22B. However, when the blood pressure exceeds a threshold (e.g., greater than about 25 mmHg, or greater than about 30 mmHg, indicative of exercise activity or stress), further expansion of the frame is limited. In an exertion-related blood pressure condition (e.g., as shown in FIGS. 21C and 22C), the blood vessel 2102 can assume an approximately circular-shaped cross-section, while the frame 2120 of flow restrictor 2100 retains an oval-shaped cross-section. In such a condition, blood flow can pass through regions 2240, which include channels (or lumen) past the flap 2110 and through the frame 2120 of flow restrictor 2100, and also include channels (or lumen) formed between frame 2120 and the blood vessel 2102. In lower blood pressure conditions, such as those shown in FIGS. 21B-21C and 22B-22C, the blood vessel 2102 and the frame 2120 do not have significant gaps between them, and the blood flow through the blood vessel 2102 primarily flows through the frame 2120.

In flow restrictor 2100, flap 2110 is tilted (biased) down under low (or normal) pressure conditions, and is configured to transition between the tilted state (relative to plane 1705 of the outflow end of frame 1720) in low (or normal) pressure ranges, and to move flap 2110 toward alignment with the outflow plane of frame 2120 in response to elevated pressures, thereby restricting blood flow. Additionally, in some cases flap 2110 and rigid push member 2130 in flow restrictor 2100 of FIGS. 21A-22C may not be configured to cause the flap 2110 to prolapse under exertion-related blood pressure conditions. The regions 2240, including those between the frame 2120 and the blood vessel 2102, can allow sufficient flow of blood under exertion-related blood pressure conditions without flap 2110 prolapsing, as shown in FIGS. 21C and 22C. In some cases, two or more flaps (similar to flaps 1910a, 1910b in FIGS. 19A-20C), or one larger flap (similar to flap 1710 in FIGS. 17A-18C), can be used to further restrict the lumen through frame 2120.

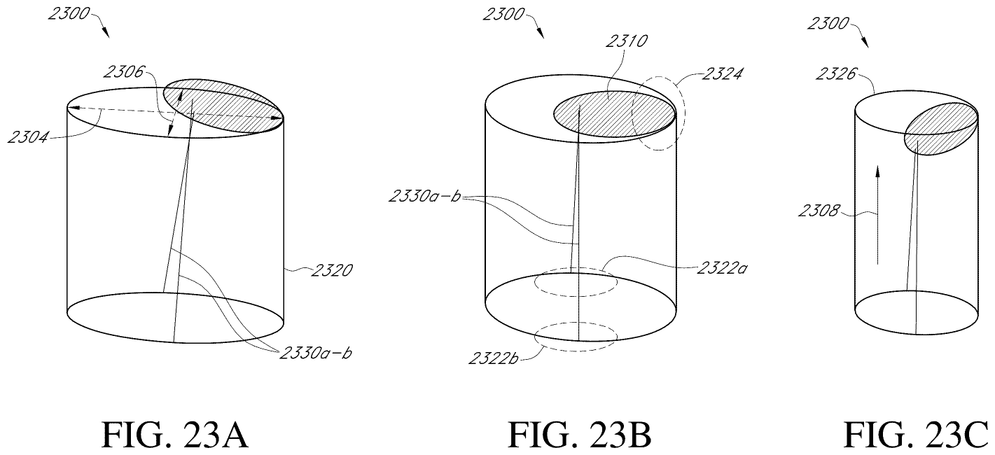

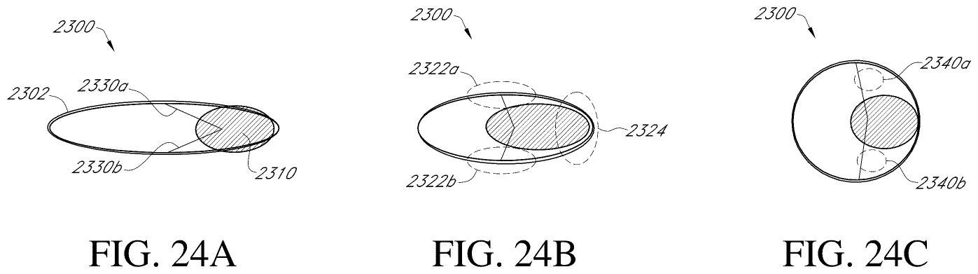

FIGS. 23A-24C show isometric and top-down views of an example flow restrictor 2300 that includes one or more flaps 2310 attached to, and movable by, two pull members 2330a, 2330b. Pull members 2330a, 2330b may be under tension during the operation of flow restrictor 2300, and therefore can be rigid or flexible. FIGS. 24A-24C show top-down views of flow restrictor 2300, in states that correspond to those shown in FIGS. 23A-23C, respectively. Flow restrictor 2300 is shown in FIGS. 23A and 24A under normal blood pressure conditions (e.g., about 1 mmHg to about 15 mmHg), in FIGS. 23B and 24B under elevated blood pressure conditions (e.g., from about 10 mmHg to about 25 mmHg, or from about 15 mmHg to about 25 mmHg, or from about 10 mmHg to about 30 mmHg), and in FIGS. 23C and 24C under exertion-related blood pressure conditions (e.g., greater than about 25 mmHg, or greater than about 30 mmHg). Flow restrictor 2300 includes an oval frame 2320 (or stent) with a flap 2310 hinged to the outflow end of oval frame 2320, similar to flap 1710 and frame 1720 described above in FIGS. 17A-18C.

FIGS. 24A-24C show both the frame 2320 and a blood vessel 2302 surrounding frame 2320. However, a cross-section of the frame 2320 is significantly oversized relative to a cross-section of blood vessel 2302 (e.g., oversized by about 10% to about 200%, about 50% to about 100%, about 100% to about 200%, etc.), and therefore, blood vessel 2302 is in contact with frame 2320 in all states shown in FIGS. 24A-24C.

In the example shown in FIGS. 23A-24C, flow restrictor 2300 includes two pull members 2330a, 2330b. Flap 2310 is coupled to frame 2320 in a region 2324 at the outflow end 2326 of frame 2320, the two pull members 2330a, 2330b (such as rods or wires) are coupled to flap 2310, and the other ends of the pull members 2330a, 2330b are coupled to frame 2320 in regions 2322a, 2322b at the inflow end of the frame 2320. The short axis 2306 of the oval cross-section intersects frame 2320 in regions 2322a and 2322b, which can be referred to as short-axis regions of frame 2320. The long axis 2304 of the oval cross-section intersects frame 2320 in region 2324, which can be referred to as a long-axis region of frame 1720. In this example, rigid pull members 2330a, 2330b are coupled to flap 2310 at a point that is a spaced away from a point where the flap 2310 is coupled to the frame 2320, in a configuration that enables pull members 2330a, 2330b to move flap 2310 when oval-shaped frame 2320 is distorted (thereby becoming more cylindrical) as shown in FIGS. 23A-24C. In some cases, two or more flaps (similar to flaps 1910a, 1910b in FIGS. 19A-20C), or one larger flap (similar to flap 1710 in FIGS. 17A-18C), can be used to further restrict the lumen through frame 2320.

FIGS. 23A and 24A show flow restrictor 2300 under normal blood pressure conditions (e.g., from about 1 mmHg to about 15 mmHg). Frame 2320 is configured to be significantly oversized (e.g., oversized by about 10% to about 200%, about 50% to about 100%, about 100% to about 200%, etc.) compared to the blood vessel 2302, and to assume an oval shape that forces a wall of the blood vessel to be similarly shaped at these normal (relatively low) blood pressures (e.g., when implanted in the IVC). Since frame 2320 is significantly oversized compared to the blood vessel 2302, the frame 2320 may be in contact with the blood vessel 2302 in all states shown in FIGS. 23A-24C. In the initial state shown in FIGS. 23A and 24A, the length of the pull members 2330a, 2330b, the size of frame 2320, and the coupling regions 2322a, 2322b and 2324 (of pull members 2330a, 2330b and flap 2310 to frame 2320, respectively) are configured to tilt the flap upwards (in the direction 2308 of the blood flow). This can be accomplished by designing the flap 2310 to naturally bias upwards (in the absence of blood flow), or by allowing it to tilt upward in response to the blood flow pushing there-against at the low (or normal) pressures.

FIGS. 23B and 24B show flow restrictor 2300 under elevated blood pressure conditions (e.g., from about 10 mmHg to about 25 mmHg, or from about 15 mmHg to about 25 mmHg). When the device is implanted in a blood vessel (e.g., the IVC, for example upstream from the renal veins), elevated venous pressure may force the frame 2320 and blood vessel 2302 to assume a less oval shape, as shown in FIGS. 23B and 24B, which may cause the pull members 2330a, 2330b to pull the flap 2310, causing the flap 2310 to move downwards (i.e., opposite the direction 2308 of the blood flow) and restrict a greater portion of the outflow, thereby restricting blood flow at higher pressures.

FIGS. 23C and 24C show flow restrictor 2300 under exertion-related blood pressure conditions (e.g., greater than about 25 mmHg, or greater than about 30 mmHg). The frame 2320 can be designed in this example to be significantly oversized relative to the blood vessel 2302, such that when the venous pressure exceeds an upper threshold (e.g., greater than about 25 mmHg, or greater than about 30 mmHg), which may be indicative of the pressures during exercise activity and/or stress, the frame 2320 and blood vessel 2302 therearound assume a circular (or nearly circular, or more circular) cross-sectional profile, which has an increased cross-sectional area relative to the oval profile. The exertion-related blood pressure can thereby cause the pull members 2330a, 2330b to further pull the flap 2310 downwards (i.e., opposite the direction 2308 of the blood flow), as shown in FIGS. 23C and 24C, such that the projected area of the flap 2310 restricts a smaller portion of the outflow, thereby allowing less restricted blood flow therethrough. Additionally, in some cases, the more circular cross-section of frame 2320 and blood vessel 2302 may allow more blood to flow around the sides of flap 2310 because flap 2310 can be approximately oval shaped, for example through regions 2340a, 2340b. When the blood pressure is lowered, the frame 2320 may reassume the more oval shape and the pull members may pull on the flap less, allowing the flap to naturally bias back upwards, shown in FIGS. 23A-23B and 24A-24B.

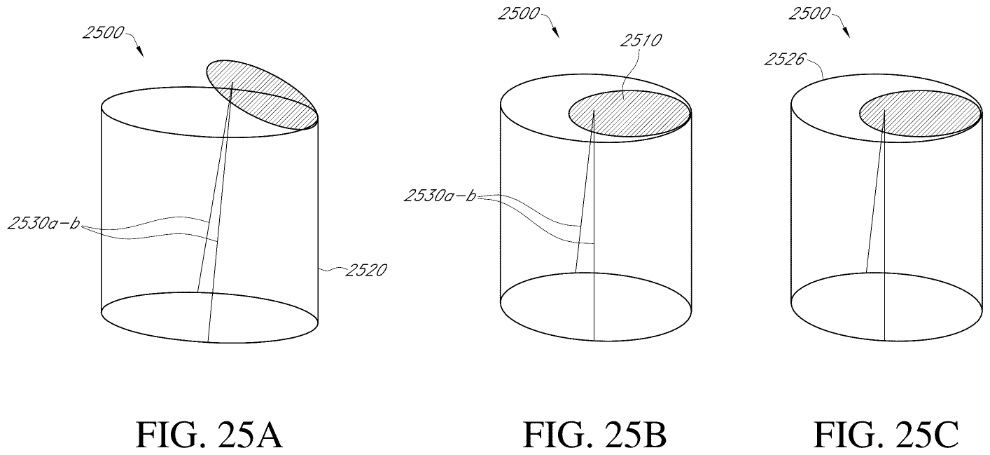

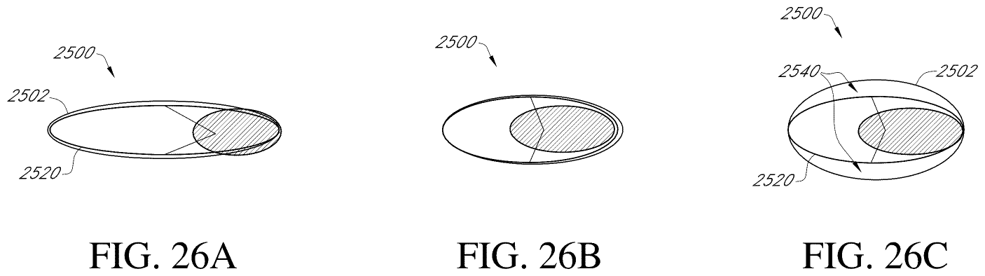

FIGS. 25A-26C show isometric and top-down views of an example flow restrictor 2500, which is similar to flow restrictor 2300 shown in FIGS. 23A-24C. However, flow restrictor 2500 has a frame 2520 that is not oversized relative to blood vessel 2502, and frame 2520 can expand from an oval shape to a less oval shape, but not necessarily to a circular (or nearly circular) profile. FIGS. 26A-26C show top-down views of flow restrictor 2500, in states that correspond to those shown in FIGS. 25A-25C, respectively. Flow restrictor 2500 is shown in FIGS. 25A and 26A under normal blood pressure conditions (e.g., about 1 mmHg to about 15 mmHg), in FIGS. 25B and 26B under elevated blood pressure conditions (e.g., from about 10 mmHg to about 25 mmHg, or from about 15 mmHg to about 25 mmHg, or from about 10 mmHg to about 30 mmHg), and in FIGS. 25C and 26C under exertion-related blood pressure conditions (e.g., greater than about 25 mmHg, or greater than about 30 mmHg).

The components and operation of flow restrictor 2500 in FIGS. 25A-26C are similar to or the same as those of flow restrictor 2300 in FIGS. 23A-24C described above, except that frame 2520 is not oversized (or is minimally oversized) relative to blood vessel 2502. Flow restrictor 2500 in FIGS. 25A-26C includes a flap 2510 attached to pull members 2530a, 2530b (e.g., wires or rods). In this example, an expansion of frame 2520 is limited when blood pressure exceeds the threshold into exertion-related blood pressure conditions (similar to flow restrictor 2100 shown in FIGS. 21C and 23C), allowing blood to flow both through the outflow end 2526 of the flow restrictor 2500 and also through channels 2540 formed between frame 2520 and blood vessel 2502, as shown in FIGS. 25C and 26C. Additionally, in some cases, flap 2510 and pull member 2530 may not be configured to cause the flap 2510 to prolapse under exertion-related blood pressure conditions. In such cases, the channels 2540 between the frame 2520 and the blood vessel 2502 can allow sufficient flow of blood under exertion-related blood pressure conditions without flap 2510 prolapsing, as shown in FIGS. 25C and 26C.

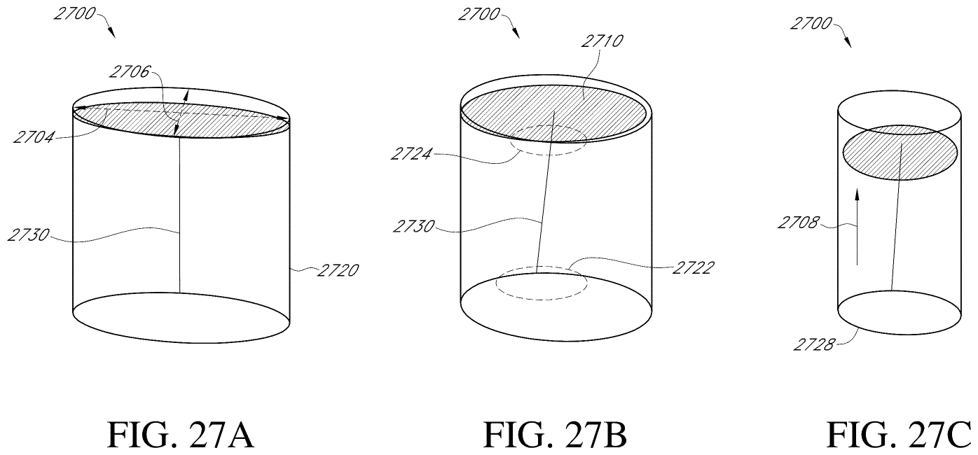

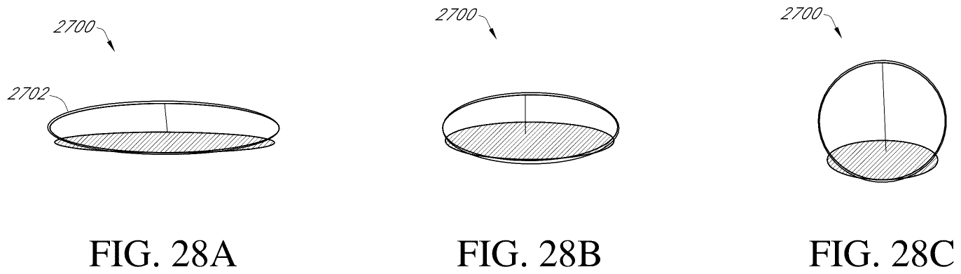

FIGS. 27A-28C show isometric and top-down views of an example flow restrictor 2700, which is similar to flow restrictor 1700 shown in FIGS. 17A-18C, but flow restrictor 2700 has a flap 2710 coupled to an outflow end of a frame 2720 at a short-axis region 2724 of frame 2720, and a rigid push member that is coupled to an inflow end 2728 of frame 2720 at a short-axis region 2722 on an opposite side of the frame 2720. The long axis 2704 and the short axis 2706 of the frame 2720 cross-section are also shown. FIGS. 28A-28C show top-down views of flow restrictor 2700, in states that correspond to those shown in FIGS. 27A-27C, respectively. Flow restrictor 2700 is shown in FIGS. 27A and 28A under normal blood pressure conditions (e.g., about 1 mmHg to about 15 mmHg), in FIGS. 27B and 28B under elevated blood pressure conditions (e.g., from about 10 mmHg to about 25 mmHg, or from about 15 mmHg to about 25 mmHg, or from about 10 mmHg to about 30 mmHg), and in FIGS. 27C and 28C under exertion-related blood pressure conditions (e.g., greater than about 25 mmHg, or greater than about 30 mmHg). The components and operation of flow restrictor 2700 are similar to or the same as those of flow restrictor 1700 in FIGS. 17A-18C described above, except that the flap 2710 and rigid push member 2730 are coupled to short-axis regions 2722 and 2724 of frame 2720, instead of long-axis regions of frame 2720. Direction 2708 shows the direction of blood flow through blood vessel 2702.

In some cases, flaps of the flow restrictors described herein (e.g., in FIGS. 1-28A) can be oval shaped, or can be shapes other than ovals, such as circles, rectangles, or irregular shapes.

While a single rod or wire is illustrated in some of the examples (such as in FIGS. 1A-6C) attached to each flap, and two wires are illustrated in other examples (such as in FIGS. 7A-10C) to be attached to each flap, it is to be understood that any flap of any flow restrictor described herein (e.g., in FIGS. 15A-28C) can be attached to a single push/pull rod or wire, two push/pull rods or wires, or any other number of push/pull rods or wires.

While a single flap is illustrated in some of the example flow restrictors described herein (e.g., those in FIGS. 17A-18C), and flow restrictors with two flaps are illustrated in other examples (such as those in FIGS. 19A-20C), it is to be understood that any of the example flow restrictors described herein (e.g., those in FIGS. 15A-28C) can include a single flap, two flaps, more than two flaps, a plurality of flaps, or any other number of flaps.

Any of the example flow restrictors disclosed above with frames including oval-shaped cross-sections (e.g., those in FIGS. 15A-28C) can be implemented either with flaps hinged to ends along the major axis or hinged to ends along the minor axis.

Any of the flow restrictors described herein (e.g., in FIGS. 1-28C) can be configured to be compatible with the IVC, the superior vena cava (SVC), or another blood vessel (e.g., a vein upstream from a renal vein). For example, any of the flow restrictors described herein (e.g., in FIGS. 1-28C) can have frames that are sized for a certain blood vessel. Any of the flow restrictors described herein (e.g., in FIGS. 1-28C) can be configured to operate in blood pressure ranges suitable for a certain blood vessel. For example, in the IVC normal (or low) blood pressure is from about 1 mmHg to about 15 mmHg, elevated blood pressure is from about 10 mmHg to about 25 mmHg, and exertion-related blood pressure is greater than about 25 mmHg or greater than about 30 mmHg. However, in other blood vessels, the normal, elevated and exertion-related blood pressure ranges can be different, and any of the flow restrictors described herein (e.g., in FIGS. 1-28C) can be configured to operate in different pressure ranges. For example, the flexibility of a leaflet, or the spring constant of a spring can be chosen to actuate a component (e.g., a leaflet, flap, or inner valve) in response to changes in blood pressures within different ranges. Moreover, any of the flow restrictors described herein (e.g., in FIGS. 1-28C) can be configured to have one mode of operation, to be bi-modal, or to have more than two modes of operation. For example, in addition to a bi-modal configuration by which the bi-modal flow restrictors described herein can adapt blood flow restriction below (e.g., less than about 25 mmHg) and above (e.g., greater than about 30 mmHg) specific pressure values, the components of the flow restrictors (e.g., flaps, wires, frame, inner valve, etc.) can be designed to provide desired restricted or unrestricted profiles at desired pressure ranges, which can differ between more than two or three pressure ranges of a bi-modal configuration.

### Methods

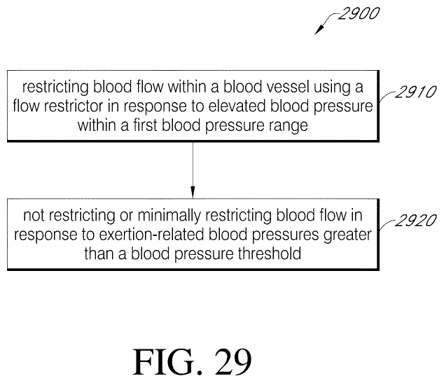

FIGS. 29-35 show examples of methods related to restricting blood flow within a blood vessel, and methods of treatment using flow modulating devices that are flow restrictors for blood vessels. The methods related to flow restrictors for blood vessels described herein can be used to occlude, partially occlude, and/or otherwise manage or regulate blood flow to or through a portion of a blood vessel (e.g., the IVC, or a blood vessel upstream from a renal vein), for example, to reduce blood pressure in blood vessels and/or organs (e.g., kidneys) downstream from the flow restrictor. The methods related to flow restrictors described herein can additionally, or alternatively, be used for any suitable applications, clinical or otherwise. For example, the methods related to flow restrictors described herein can be used for any suitable application requiring restriction of blood flow within a blood vessel, for example, to increase blood flow through an organ (e.g., the kidneys), to modulate pressure in the right atrium of the heart, and/or to reduce the accumulation of blood in the venous system.

In some examples, a method of restricting blood flow within a blood vessel includes restricting blood flow within a blood vessel using a flow restrictor described herein (e.g., in FIGS. 1-28C), in response to elevated blood pressure.

FIG. 29 shows a flowchart of an example method 2900 of restricting blood flow within a blood vessel comprising the following steps. In step 2910, blood flow is restricted within a blood vessel using a flow restrictor described herein (e.g., in FIGS. 1-28C), in response to elevated blood pressure within a first blood pressure range (e.g., from about 10 mmHg to about 25 mmHg, or from about 15 mmHg to about 25 mmHg). In step 2920, blood flow is not restricted or is minimally restricted (permitting a larger amount of blood flow) through the blood vessel in response to exertion-related blood pressures greater than a blood pressure threshold (e.g., greater than about 25 mmHg, or greater than about 30 mmHg).

In some examples, a method of treatment for a subject with congestive heart failure (CHF) and/or chronic kidney disease (CKD), includes restricting blood flow within a blood vessel using a flow restrictor described herein (e.g., in FIGS. 1-28C), in response to elevated blood pressure.

In some examples, method 2900 can be a method of treatment for a subject with congestive heart failure (CHF) and/or chronic kidney disease (CKD), comprising, in step 2910, restricting blood flow within a blood vessel using a flow restrictor described herein (e.g., in FIGS. 1-28C), in response to elevated blood pressure within a first blood pressure range (e.g., from about 10 mmHg to about 25 mmHg, or from about 15 mmHg to about 25 mmHg), and, in step 2920, blood flow is not restricted or is minimally restricted (permitting a larger amount of blood flow) through the blood vessel in response to exertion-related blood pressures greater than a blood pressure threshold (e.g., greater than about 25 mmHg, or greater than about 30 mmHg).

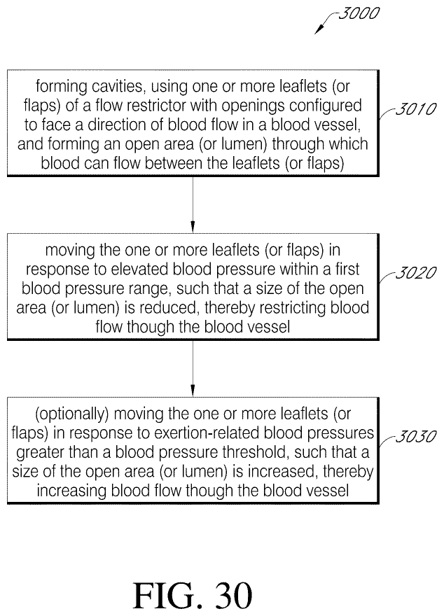

FIG. 30 shows a flowchart of an example method 3000 of restricting blood flow within a blood vessel including the following steps. In step 3010, cavities are formed, using one or more leaflets (or flaps) of a flow restrictor with openings configured to face a direction of blood flow in a blood vessel, and forming an open area (or lumen) through which blood can flow between the leaflets (or flaps) (e.g., as shown in the flow restrictors in FIGS. 1-14B). In step 3020, the one or more leaflets (or flaps) move in response to elevated blood pressure within a first blood pressure range (e.g., from about 10 mmHg to about 25 mmHg, or from about 15 mmHg to about 25 mmHg), such that a size of the open area (or lumen) is reduced, thereby restricting blood flow though the blood vessel. In optional step 3030, the one or more leaflets (or flaps) move in response to exertion-related blood pressures greater than a blood pressure threshold (e.g., about 25 mmHg or about 30 mmHg), such that a size of the open area (or lumen) is increased, thereby increasing blood flow though the blood vessel. The leaflets (or flaps) can move in steps 3020 and 3030 as described with respect to the flow restrictors described herein, for example those in FIGS. 1-14B. For example, the leaflets can prolapse in optional step 3030, such that a size of the open area (or lumen) is increased. Single mode flow restrictors may only be able to perform steps 3010 and 3020, while bi-modal flow restrictors can perform steps 3010, 3020 and 3030.

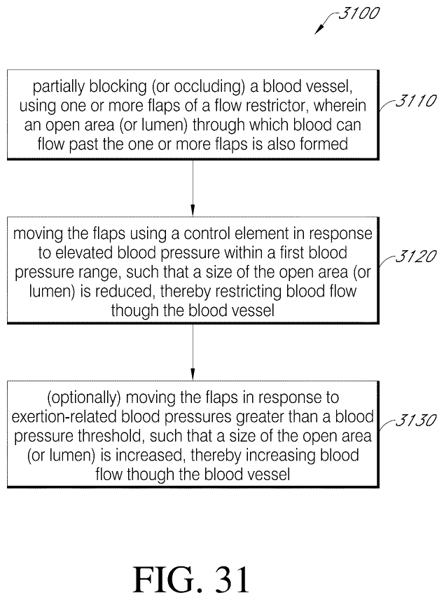

FIG. 31 shows a flowchart of an example method 3100 of restricting blood flow within a blood vessel including the following steps. In step 3110, a blood vessel is partially blocked (or occluded) using one or more flaps of a flow restrictor, wherein an open area (or lumen) through which blood can flow past the one or more flaps is also formed (e.g., as shown in the flow restrictors in FIGS. 15A-28C). In step 3120, the one or more flaps move due to action by a control element (e.g., a rigid push member, or a rigid or flexible pull member) in response to elevated blood pressure within a first blood pressure range (e.g., from about 10 mmHg to about 25 mmHg, or from about 15 mmHg to about 25 mmHg), such that a size of the open area (or lumen) is reduced, thereby restricting blood flow though the blood vessel. In optional step 3130, the one or more flaps move in response to exertion-related blood pressures greater than a blood pressure threshold (e.g., about 25 mmHg or about 30 mmHg), such that a size of the open area (or lumen) is increased, thereby increasing blood flow though the blood vessel. The flaps can move in steps 3120 and 3130 as described with respect to the flow restrictors described herein, for example those in FIGS. 15A-28C. For example, the leaflets can prolapse in optional step 3030, such that a size of the open area (or lumen) is increased. Single mode flow restrictors may only be able to perform steps 3110 and 3120, while bi-modal flow restrictors can perform steps 3110, 3120 and 3130.

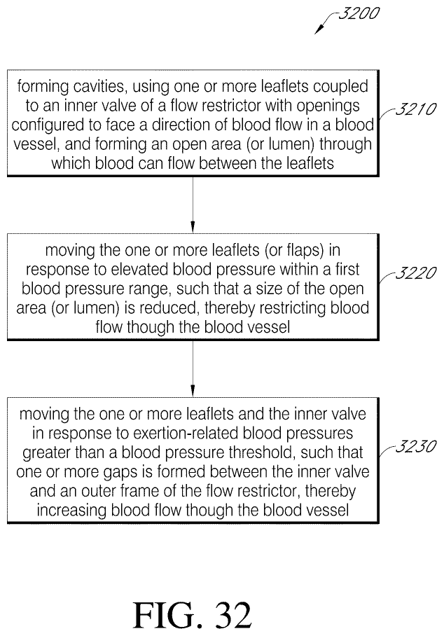

FIG. 32 shows a flowchart of an example method 3200 of restricting blood flow within a blood vessel including the following steps. In step 3210, cavities are formed, using one or more leaflets (or flaps) coupled to an inner valve of a flow restrictor, where the openings are configured to face a direction of blood flow in a blood vessel (e.g., as shown in the flow restrictors in FIGS. 5-14B). In step 3210, an open area (or lumen) is also formed, through which blood can flow between the leaflets (or flaps). In step 3220, the one or more leaflets move in response to elevated blood pressure within a first blood pressure range (e.g., from about 10 mmHg to about 25 mmHg, or from about 15 mmHg to about 25 mmHg), such that a size of the open area (or lumen) is reduced, thereby restricting blood flow though the blood vessel. In step 3230, the one or more leaflets and the inner valve move in response to exertion-related blood pressures greater than a blood pressure threshold (e.g., about 25 mmHg or about 30 mmHg), such that one or more gaps is formed between the inner valve and an outer frame of the flow restrictor, thereby increasing blood flow though the blood vessel. The leaflets (or flaps) and the inner valve can move in steps 3220 and/or 3230 as described with respect to the flow restrictors described herein, for example those in FIGS. 5-14B. For example, the inner valve can be supported by inflow support portions that rest on a seat of the outer frame, and an outflow flared portion can seal against the seat (e.g., as shown in the flow restrictor in FIG. 7). In this example, in step 3230, when the inner valve moves, it can move axially such that the one or more gaps are formed between the outflow flared portion and the seat (e.g., as shown in FIG. 9F). Additionally, the leaflets can prolapse in optional step 3230, such that a size of the open area (or lumen) is increased. Single mode flow restrictors may only be able to perform steps 3210 and 3220, while bi-modal flow restrictors can perform steps 3210, 3220 and 3230.

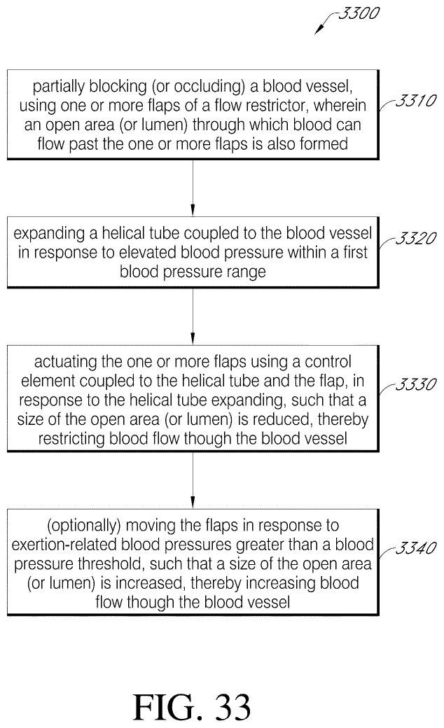

FIG. 33 shows a flowchart of an example method 3300 of restricting blood flow within a blood vessel including the following steps. In step 3310, a blood vessel is partially blocked (or occluded) using one or more flaps of a flow restrictor, wherein an open area (or lumen) through which blood can flow past the one or more flaps is also formed (e.g., as shown in the flow restrictors in FIGS. 15A-16B). In step 3320, a helical tube coupled to the blood vessel expands in response to elevated blood pressure within a first blood pressure range (e.g., from about 10 mmHg to about 25 mmHg, or from about 15 mmHg to about 25 mmHg). In step 3330, the one or more flaps are actuated using a control element coupled to the helical tube and the flap, in response to the helical tube expanding, such that a size of the open area (or lumen) is reduced, thereby restricting blood flow though the blood vessel (e.g., as shown in the flow restrictors in FIGS. 15A-16B). In optional step 3340, the one or more flaps move in response to exertion-related blood pressures greater than a blood pressure threshold (e.g., about 25 mmHg or about 30 mmHg), such that a size of the open area (or lumen) is increased, thereby increasing blood flow though the blood vessel. The flaps can move in steps 3320 and 3330 as described with respect to the flow restrictors described herein, for example those in FIGS. 15A-16B. For example, the leaflets can prolapse in optional step 3330, such that a size of the open area (or lumen) is increased. Single mode flow restrictors may only be able to perform steps 3310, 3120 and 3130, while bi-modal flow restrictors can perform steps 3310, 3320, 3330 and 3340.

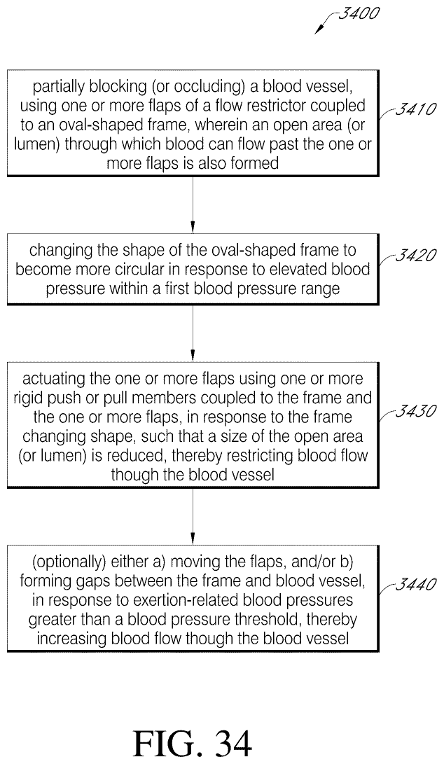

FIG. 34 shows a flowchart of an example method 3400 of restricting blood flow within a blood vessel including the following steps. In step 3410, a blood vessel is partially blocked (or occluded) using one or more flaps of a flow restrictor coupled to an oval-shaped frame, wherein an open area (or lumen) through which blood can flow past the one or more flaps is also formed (e.g., as shown in the flow restrictors in FIGS. 17A-28C). In step 3420, a shape of the oval-shaped frame is changed to become more circular in response to elevated blood pressure within a first blood pressure range (e.g., from about 10 mmHg to about 25 mmHg, or from about 15 mmHg to about 25 mmHg). In step 3430, the one or more flaps are actuated using one or more rigid push or pull members coupled to the frame and the one or more flaps, in response to the frame changing shape, such that a size of the open area (or lumen) is reduced, thereby restricting blood flow though the blood vessel (e.g., as shown in the flow restrictors in FIGS. 17A-28C). In optional step 3340, either a) moving the flaps, and/or b) forming gaps between the frame and the blood vessel, in response to exertion-related blood pressures greater than a blood pressure threshold (e.g., about 25 mmHg or about 30 mmHg), thereby increasing blood flow though the blood vessel. The flaps can move in steps 3420 and 3430 as described with respect to the flow restrictors described herein, for example those in FIGS. 17A-28C. For example, the frame can be configured to maintain an oval-shaped cross-section at exertion-related blood pressures and the blood vessel can have a circular cross-section at exertion-related blood pressures, which can cause gaps to form between the frame and the blood vessel through which blood can flow (e.g., as shown in FIGS. 22C and 26C). Single mode flow restrictors may only be able to perform steps 3410, 3420 and 3430, while bi-modal flow restrictors can perform steps 3410, 3420, 3430 and 3440.

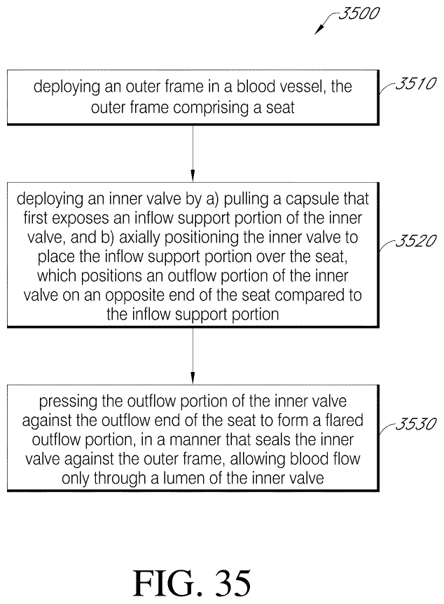

FIG. 35 shows a flowchart of an example method 3500 of deploying a flow restrictor within a blood vessel, wherein the flow restrictor is configured to restrict blood flow within the blood vessel, including the following steps. In step 3510, an outer frame is deployed in a blood vessel, the outer frame comprising a seat (e.g., as shown in FIG. 9B). In step 3520, an inner valve is deployed by pulling a capsule that first exposes an inflow support portion of the inner valve; and axially positioning the inner valve to place the inflow support portion over the seat, which positions an outflow portion of the inner valve on an opposite end of the seat compared to the inflow support portion (e.g., as shown in FIG. 9C). In step 3530, the outflow portion of the inner valve is pressed against the outflow end of the seat to form a flared outflow portion, in a manner that seals the inner valve against the outer frame, allowing blood flow only through a lumen of the inner valve (e.g., as shown in FIG. 9D).

### Example Implantation of Flow Modulating Devices

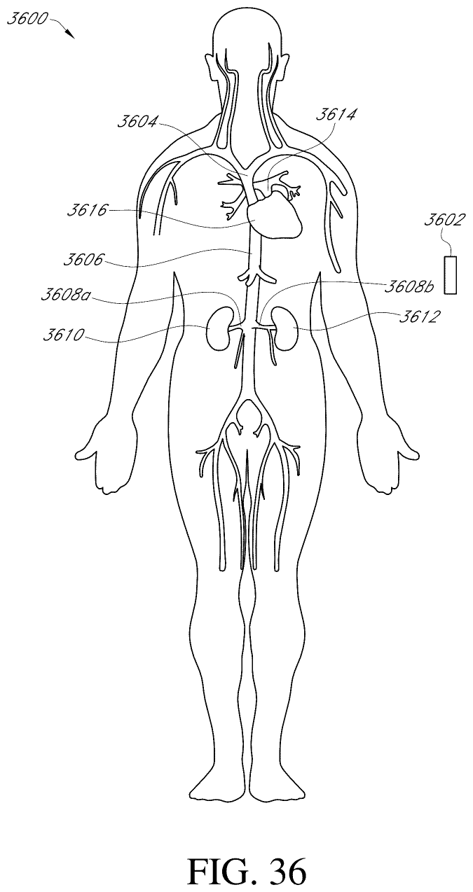

FIG. 36 illustrates a schematic representation of portions of a subject 3600. The flow modulating devices described herein (represented in FIG. 36 by device 3602) may be introduced (e.g., implanted) in vasculature of the body. In general, the device 3602 may represent any of the flow modulating devices described herein (e.g., flow restrictors described with respect to those shown in FIGS. 1-28A) and may include the same or similar functionality and/or structures. In some examples, the device 3602 may be implanted in or near to a portion of the Superior Vena Cava (SVC) 3604. In some examples, the device 3602 may be implanted in or near to a portion of the Inferior Vena Cava (IVC) 3606. The subject 3600 is illustrated with a representation of a portion of the vasculature system to generally illustrate the SVC 3604 and the IVC 3606 within the subject 3600. However, it is to be understood that no dimensions or relative sizes of components may be inferred from the relative sizes and dimensions of elements in the figures.

The subject 3600 includes a number of vessels and organs that may circulate blood throughout the body. For example, renal veins 3608a and 3608b drain blood from respective right kidney 3610 and left kidney 3612. Renal veins 3608a and 3608b connect to the IVC 3606. Blood from the aorta 3614 flows to the IVC 3606. Blood travels from the aorta 3614 to the abdominal organs including the stomach (not shown), liver (not shown), spleen (not shown), pancreas (not shown), large intestines (not shown), and small intestine (not shown). Following processing of the blood by the liver, blood collects in the central vein. Blood from these central veins converges in the hepatic veins (not shown) which exit the liver and empty into the IVC 3606 to be distributed to the rest of the body.

Portions of the above-recited blood circulating vessels and/or organs may be involved in splanchnic venous circulation that includes blood flow originating from the celiac, superior mesenteric, and inferior mesenteric arteries to the abdominal organs. The splanchnic venous circulation may act as a blood reservoir that can support the need for increased stressed blood volume during periods of elevated sympathetic tone, such as during exertion, to support increased cardiac output and vasodilation of peripheral vessels supporting active muscles.

However, heart failure patients can have multiple comorbidities that prevent the use of this additional blood reservoir. Example comorbidities can include chronic kidney disease, chronotropic incompetence, inability to increase stroke volume, and/or peripheral microvascular dysfunction. This can lead to venous congestion and/or abrupt rises in central venous pressure, pulmonary artery pressure, and/or pulmonary capillary wedge pressure. To alleviate such pressures, the blood reserves within the blood reservoir described above can be used to support the need for increased stressed blood volume during periods of elevated sympathetic tone. The flow modulating devices described herein may be used to ensure that such blood reserves within the blood reservoir can be utilized.

For example, because blood flow from the splanchnic venous circulation is directed through hepatic veins and into the IVC 3606, devices (as described herein) may be placed into the IVC 3606 to limit blood flow to allow the reservoir to expand with increased blood volume. Similarly, devices (as described herein) may be placed into the SVC 3608 to limit blood flow to allow the reservoir to expand with increased blood volume. Furthermore, the flow modulating devices described herein may be placed in either the IVC 3606 and/or SVC 3608 to alleviate pressure in the right side of the atrium of the heart 3616.

In some examples, the flow modulating device 3602 (representing the devices described herein) may be used as a method of treatment to treat any combination of heart failure, chronic kidney disease, chronotropic incompetence, inability to increase stroke volume, and/or peripheral microvascular dysfunction. In addition, the flow modulating device 3602 may be used as a method of treatment to regulate pressure in the right atrium of the heart. Further, the flow modulating device 3602 may be used as a method of treatment to improve function of the kidneys in patients having reduced kidney function due to pressure in the venous system.

In some embodiments, a method of treatment can include using one or more methods and/or devices described herein to facilitate management of blood flow through and/or into one or more blood vessels and/or chambers of a heart. There is a need for new and useful method of treatment using flow restrictors for blood vessels. In particular, there is a need for methods of treatment using the systems, devices, and methods described herein, the methods including modulating and/or balancing of blood flow through a blood vessel, for example, to occlude, partially occlude, and/or otherwise manage or regulate blood flow to or through a portion of a blood vessel.

In some embodiments, a method of treatment for restricting blood flow within a blood vessel includes using a flow restrictor for a blood vessel, wherein the flow restrictor includes a frame positionable within a blood vessel, and two or more leaflets each comprising an inflow end and an outflow end. The outflow end of each leaflet can be coupled to the frame, and the inflow end of each leaflet can be movable in a radial direction, wherein the radial direction is approximately perpendicular to a central axis of the frame. Each leaflet can define a cavity having an opening that is configured to face a direction of blood flow within the blood vessel. The inflow end of each leaflet can be configured to move toward each other in response to a first increase in a blood pressure within the blood vessel, and in response to the blood pressure within the blood vessel being within a first blood pressure range.

In some embodiments, a method of treatment for restricting blood flow within a blood vessel includes using a bi-modal flow restrictor for a blood vessel, wherein the bi-modal flow restrictor includes: an outer frame positionable within a blood vessel; an inner valve positioned within the outer frame; two or more leaflets each comprising an inflow end and an outflow end; and a first spring positioned between the inner valve and the outer frame. The inner valve can be movable in an axial direction within the outer frame, and the axial direction is approximately parallel with a central axis of the outer frame. The outflow end of each leaflet can be coupled to the inner valve, the inflow end of each leaflet can be movable in a radial direction, wherein the radial direction is approximately perpendicular to the central axis of the outer frame. Each leaflet can define a cavity having an opening that is configured to face a direction of blood flow within the blood vessel, and the inflow end of each leaflet can be configured to move toward each other in response to a first increase in a blood pressure within the blood vessel, and in response to the blood pressure within the blood vessel being within a first blood pressure range. The first spring can be configured to hold the inner valve in a first axial position within the outer frame when the blood pressure within the blood vessel is within the first blood pressure range, and to allow the inner valve to move to a second axial position when the blood pressure within the blood vessel is within a second blood pressure range, wherein the second axial position is downstream from the first axial position. A lower limit of the second blood pressure range can be higher than an upper limit of the first blood pressure range. An outflow ring of the inner valve can be pressed against a seat of the outer frame when the inner valve is in the first axial position, and the outflow ring of the inner valve can be configured to move away from the seat of the outer frame when the inner valve is in the second axial position thereby allowing the blood flow to bypass the inner valve and flow between the inner valve and the outer frame.

In some embodiments, a method of treatment for restricting blood flow within a blood vessel includes using a bi-modal flow restrictor for a blood vessel, wherein the bi-modal flow restrictor includes an outer docking frame positionable within a blood vessel, and an inner valve positioned within the outer docking frame. The inner valve can be configured to be sealed against the outer docking frame when blood pressure within the blood vessel is within a first blood pressure range. The inner valve can include two or more leaflets configured to move closer to each other in response to elevated pressure within the blood vessel and restrict flow through the inner valve when the inner valve is sealed against the outer docking frame. The inner valve and the outer docking frame can be configured such that a gap is formed between the inner valve and the outer docking frame when blood pressure within the blood vessel is within a second blood pressure range, wherein a lower limit of the second blood pressure range is higher than an upper limit of the first blood pressure range. Blood can flow through the gap between the inner valve and the outer docking frame when the gap is formed.

In some embodiments, a method of treatment for restricting blood flow within a blood vessel includes using a flow restrictor for a blood vessel, wherein the flow restrictor includes: a frame positionable within a blood vessel; a first flap comprising a first flap inflow end and a first flap outflow end; a first control element coupled to the first flap; a first helical tube coupled to a wall of the blood vessel; and a first guide tube coupled to the frame. The first flap inflow end can be coupled to the frame such that the first flap inflow end is movable in a radial direction, wherein the radial direction is approximately perpendicular to a central axis of the frame. The first control element can be fixed to a first end of the first helical tube, can extend through the first helical tube, can exit the first helical tube at a second end, can pass through the first guide tube, and can be fixed to the first flap outflow end. The first helical tube can be configured to expand in response to an enlargement of the blood vessel, and the first control element can be configured to pull the first flap radially inward in response to the expansion of the first helical tube.

In some embodiments, a method of treatment for restricting blood flow within a blood vessel includes using a flow restrictor for a blood vessel, wherein the flow restrictor includes: a frame configured to be coupled to a wall of a blood vessel; a first flap coupled to an outflow end of the frame using a first coupling such that the first flap is movable about the first coupling; and a first member coupled to an inflow end of the frame and to the first flap. A cross-section of the frame approximately perpendicular to a central axis of the frame can be approximately oval-shaped. The first member can have an approximately constant length. In response to an increase in blood pressure, the cross-sectional area of the frame can be configured to increase by assuming a less oval cross-sectional shape approximately perpendicular to the central axis of the frame, and the first member can be configured to move the first flap in response to the frame assuming the less oval cross-sectional shape.

As used herein, the term “active” with respect to blood flow management may represent operations carried out by the devices described herein using power or controller induced movement. For example, actively moving a portion of the devices described herein may include the use of battery power, wall outlet power, magnetic field induction, electromagnetic field induction, magnetic polarization, a piston-based system, a valve-based system (e.g., with a manifold), hydraulics, pneumatics, optical actuators, thermal actuators, and/or other actuator using electrical or inductive power.

In some implementations, an active control mechanism may include a microcontroller and/or a power source implanted with or integrated with the flow management device. Alternatively, or additionally, an active control mechanism can include a microcontroller and/or a power source in a remote control device, external to the body, or in an implanted remote device (e.g., subcutaneously, intravascularly, etc.), for example. The remote control device may be in wireless communication with the implanted device or connected to the implanted device through one or more leads.

In any of the embodiments described herein, an active mechanism may include a pump fluidly connected to a reservoir; a chamber having a first portion and a second portion; a manifold fluidly connected to the pump, the reservoir, and the chamber; and a piston coupled to a control element of a flow modulating device. The manifold may include at least one port that fluidly connects the reservoir to the first portion of the chamber. The piston can move between a restricted blood flow position and an unrestricted blood flow position within the chamber, any position therebetween for intermediate blood flow restriction positions. For example, the piston may move to the restricted blood flow position when a fluid is flowed from the reservoir, charged by a pump, through the manifold into the first portion of the chamber. The piston can return to the unrestricted blood flow position when the fluid is evacuated from the first portion of the chamber. In some embodiments, the manifold is fluidly connected to a second portion of the chamber through a second port. In such embodiments, the piston can move to the unrestricted blood flow position when the fluid enters the second port from the reservoir through the manifold, thereby causing the valve of the flow modulating device to move to the unrestricted blood flow state. In some embodiments, the fluid is evacuated from the second portion of the chamber through the second port when the piston is in the restricted blood flow position. In some implementations, the at least one port further fluidly connects the first portion of the chamber to the pump through the manifold. For example, the at least first port is fluidly connected to the pump through the manifold to evacuate the fluid from the first portion of the chamber thereby moving the piston to the unrestricted blood flow position. In some examples, the piston is a spring-based piston. For example, the spring-based piston can automatically return to the unrestricted blood flow position when the fluid is evacuated from the first portion of the chamber.

In any of the embodiments described herein, an active mechanism may include a linear actuator coupled to a control element of the flow management device. The linear actuator tensions the control element to position the valve of the flow management device in the restricted blood flow state. Alternatively, the linear actuator releases tension in the control element to position the valve in the unrestricted blood flow state. The tensioning and releasing of tension on the control element may be based on a predefined set of parameters or based on a sensed attribute of the blood vessel in which the flow management device is implanted. For example, the sensed attribute may be sensed by a sensor. The sensor may be coupled to the flow management device, a remote control device, or otherwise in wireless or electrical communication with a flow management system. The sensor can be a strain gauge, a piezoelectric sensor, a capacitance sensor, or a vacuum pressure sensor, such that the sensor senses a pressure in the blood vessel.

In any of the embodiments described herein, the linear actuator is an electromechanical linear actuator having a first magnet that, when caused to rotate by another magnet or actuator, causes a nut to rotate on a lead screw, the nut being coupled to the control element. A second magnet in a control device may cause rotation of the first magnet, for example by changing its magnetic field pole direction. In some embodiments, a repeater magnet (with or without its own power source) is positioned between the first magnet and the second magnet, for example in cases where the first magnet is beyond a threshold distance from the second magnet.

In any of the embodiments described herein, the linear actuator is a pneumatic linear actuator having a piston coupled to the control element. Injecting compressed gas moves the piston to tension the control element to move the valve into a restricted blood flow state and venting the compressed gas releases tension in the control element to move the valve to an unrestricted blood flow state.

In any of the embodiments described herein, the linear actuator is a hydraulic linear actuator having a piston coupled to the control element. Injecting liquid moves the piston to tension the control element to move the valve into a restricted blood flow state and venting the liquid releases tension in the control element to move the valve to an unrestricted blood flow state.

In any of the embodiments described herein, the linear actuator is a thermal linear actuator having a piston coupled to the control element. For example, increasing a temperature of a thermal sensitive fluid (e.g., via a heat source, changes in body temperature, etc.) causes the piston to compress the fluid to tension the control element to move the valve into the restricted blood flow state. Alternatively, decreasing the temperature of the thermal sensitive fluid causes the piston to decompress the fluid to release tension in the control element to move the valve to the unrestricted blood flow state.

As used herein, the term “passive” with respect to blood flow management may represent operations carried out by the devices described herein using passively induced movement. For example, passively moving a portion of the devices described herein may include the use of manual pull wires (e.g., sutures, actuation wires/cords, etc.), anatomy responses (e.g., changes in vessel inner diameter, intra-vessel pressure, etc.), blood movement, or the like.

In some examples described herein, a passive mechanism can include a control element, such as a rigid push or pull member (e.g., a rod or rigid wire), a flexible tension member, a flexible wire, a suture, a string, a cable, or the like. One or more control elements can be coupled to a body to be moved (or actuated), such as one or more leaflets, flaps, valves, or valve portions (e.g., used to restrict blood flow through a blood vessel) and to a frame (or other component). For example, the control element can be coupled to a frame that moves in response to an external force (e.g., an increase in blood pressure). In some examples, the control element is coupled to a helical tube that expands and contracts with a blood vessel in which it is positioned, and the movement of the helical tube moves the control element. In some examples, a control member can be used to actuate the valve in response to the movement of the frame (or helical tube) due to a change in blood pressure within a blood vessel.

In some examples described herein, a passive mechanism can include a spring, or elastic member (such as a flexible commissure). One or more springs or elastic members can be coupled to a body to be moved (or actuated), such as one or more leaflets, flaps, valves, or valve portions (e.g., used to restrict blood flow through a blood vessel) and to a frame. For example, a spring can be used to bias the leaflet, flap, or valve in an initial position (e.g., under relatively lower blood pressure conditions). An externally applied force can cause the leaflet, flap, or valve to move and extend the spring (e.g., under relatively higher blood pressure conditions, for example wherein increased blood flow applies pressure against the leaflet, flap, or valve). In the absence of the applied force, the spring can recompress, thereby bringing the leaflet, flap, or valve back to its initial position.

In some examples described herein, a passive mechanism can include more than one mode of operation. For example, a body (or member or component) of a blood flow regulator (or restrictor) described herein can move in a particular way in response to a first externally applied force, and then the body (or member or component) of a blood flow regulator (or restrictor) described herein can move in a different way in response to a second externally applied force. In some cases, the first externally applied force and the second externally applied force are applied from the same external force (e.g., blood pressure), with different quantitative ranges. For example, a leaflet, flap, or valve (e.g., used to restrict blood flow through a blood vessel) can move in a first mode in response to an increase in blood pressure within a first blood pressure range, and move in a second mode in response to an increase in blood pressure within a second blood pressure range that is different (e.g., higher) than the first blood pressure range. The first mode can be that an end of the leaflets, flaps, or valve portions moves towards one another to further restrict blood flow within the blood vessel, and the second mode can be that the leaflets, flaps, or valve portions prolapse, thereby moving away from one another to increase the blood flow within the blood vessel.

In some examples described herein, a flow modulating device (e.g., a flow restrictor) described herein can include more than one passive mechanism. For example, a flow modulating device can contain a first passive mechanism that can move in a first mode in response to an increase in blood pressure within a first blood pressure range, and a second passive mechanism that can move in a second mode in response to an increase in blood pressure within a second blood pressure range that is different (e.g., higher or maximum) than the first blood pressure range. The first passive mechanism can include leaflets, flaps, or valve portions that move towards one another to further restrict blood flow within the blood vessel, and the second passive mechanism can include an inner valve that can move (e.g., axially) to open additional channels through the flow regulator device to increase the blood flow. In some cases, the inner valve can be biased using a passive element such as one or more springs or spring like elements, such that it may return to its initial position after the blood pressure decreases (e.g., back into a first blood pressure range).

Any of the implantable or flow modulating devices described herein may be coated with a polymer (e.g., silicones, poly(urethanes), poly(acrylates), or copolymers such as poly(ethylene vinyl acetate), a drug (e.g., heparin, pro-endothelialization drugs, anti-thrombogenic drug, etc.), a textile (e.g., woven, knitted, nonwoven, or braided), tissue (e.g., bovine pericardium, equine pericardium, porcine vena cava, etc.), or a combination thereof. Woven and knitted fabrics may be made from poly (ethylene terephthalate), while the nonwoven fabrics may be made from expanded poly(tetrafluoroethylene). Some textiles may also or alternatively include silk or silk-based materials.

Further, any of the pull wires, sutures, or actuation wires described herein may include silk, silk-based materials, nylon, synthetic polymer materials (e.g., silicone, polydioxanone, polyglycolic acid, polyglyconate, polylactic acid, etc.), natural materials (e.g., purified catgut, collagen, sheep intestines, cow intestines, etc.), metal (e.g., Nitinol, palladium, gold and their alloys, etc.), or a combination thereof.

The flow modulating devices described herein may be part of (or installed within) a stent. The stent may represent a frame or outer frame that provides a support structure for the flow modulating devices when the stent is implanted into a blood vessel. The frame/outer frame may be a self-expanding frame or a balloon-expandable frame. In general, any type of stent may be used with the flow modulating devices. Example stents may include, but are not limited to, bare metal stents, coated stents, drug-eluting stents, biodegradable stents, balloon expandable stents, and self-expandable stents.

The stents described herein may be configured to house all or a portion of the flow modulating devices described herein. Such stents may include an assembly with strut members interconnected by joints that form a series of linked mechanisms that result in a hollow tube-shaped element. The stents may be positioned and/or repositioned within a blood vessel to introduce or remove flow modulating devices or device members including, but not limited, to valving, control elements, balloons, flexible members, rigid members, adjustment mechanisms, sensors, coils, wires, and/or magnets. One or more of such device members may be actuated to modify stent shape (or device member shape) for purposes of modifying a flow of fluid through the vessel associated with the implanted stent. Moreover, the stents described herein may partially or fully surround a flow modulating device. For example, a stent or stent portion may surround a portion of a flow modulating device to ensure the device remains in a specified position in a blood vessel. In some examples, the stent surrounds the flow modulating device entirely. In some examples, the stent surrounds the flow modulating device and further continues beyond one or both ends of the device.

The stents described herein may include an outer frame. The outer frame may have a form and structure that varies. For example, the strut members and/or articulated joints may form a mesh-like structure. The strut members may be interconnected in such a way as to form a shaped pattern of cells. For example, any number of strut members may form a ring of the stent such that the strut members are connected by any number of crowns. Any number of rings may form a body of the stent, and the rings may be connected by any number of bridges. Example cell shapes may include, but are not limited to diamond, square, rectangle, triangle, oval, ganglion, or any combination thereof. In some examples, the cells may be evenly shaped and distributed from a first end of the stent to a second end of the stent. In some examples, the cells may include a number of strut members interconnected in such a way that when the stent expands radially, one or more of the cells become longitudinally shorter. Similarly, when the stent constricts radially, one or more of the cells become longitudinally longer.

Constricting portions of the stents described herein may result in an outer frame woven tighter than other portions of the stent that are not constricted. The constriction may push against one or more portions of the flow modulating devices described herein to narrow a pathway through the frame or outer frame and/or to trigger the flow modulating device to begin or end constriction. Similarly, expanding portions of the stents described herein may result in an outer frame woven looser than other portions of the stent that are not expanded. The expansion may release one or more portions of the flow modulating devices described herein to widen a pathway through the frame or outer frame and/or to trigger the flow modulating device to begin or end constriction.

The flow modulating devices described herein may be introduced to a vessel or tissue site using a delivery system. For example, such delivery systems may be used to position catheter tips and/or catheters in various portions of a target vasculature. A delivery system may include a delivery catheter having a pusherwire or the like disposed therein. The pusherwire may be configured to deploy any of the devices described herein, for example by urging the device out of a distal end of the catheter and either actively expanding the device or allowing the device to passively expand once it is no longer constrained by a lumen of the catheter. Any of the devices described herein may be crimped or otherwise compressed such that a cross-sectional area of the device is sized and/or shaped to be delivered through a lumen of a catheter. In some examples, the crimped or compressed device may be transferred to the delivery system using a transfer sheath, or the like. A delivery system can access the vasculature through an access site, such as a radial artery, brachial artery, internal jugular vein, common femoral vein, subclavian veins, or the like.

For example, in a coronary procedure, a catheter tip and/or catheter may be configured to pass from the right atrium into the coronary sinus. For access to the venous circulation, for example, a catheter tip and/or catheter may be configured to pass from the radial artery into the superior vena cava. Further, for central venous access, a catheter tip and/or catheter may be configured to pass from the femoral vein into the inferior vena cava.

In some examples, the delivery system may include a trocar or other suitable delivery device may be used for implanting devices subcutaneously, for example control devices for controlling activation of any of the flow modulating devices described herein. As described elsewhere herein, various control systems may include an implanted remote device that is configured to transmit control signals to a flow modulating device disposed in the vasculature. The control signals may include signals transmitted wirelessly, through a wired connection (e.g., leads), or via magnetic field induction, electromagnetic field induction, or magnetic polarization.

However, it may be understood that the delivery system can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels (e.g., superior vena cava, inferior vena cava, renal artery, renal vein, etc.), and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device including an inner lumen configured to slidably receive instrumentation, such as for positioning within an atrium, coronary sinus, superior vena cava, or inferior vena cava, including for example delivery catheters, cannulas, and/or trocars. It may be understood that other types of medical implant devices and/or procedures can be delivered to the coronary sinus, superior vena cava, inferior vena cava, etc. using a delivery system as described herein, including for example ablation procedures, drug delivery, and/or placement of actuator leads.

Described herein are various example medical implants and/or delivery methods. Some examples described herein may be used in combination and/or may be used independently.

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A flow restrictor for a blood vessel, comprising: a frame positionable within a blood vessel; and two or more leaflets each comprising an inflow end and an outflow end, wherein the outflow end of each leaflet is coupled to the frame, wherein the inflow end of each leaflet is movable in a radial direction, wherein the radial direction is approximately perpendicular to a central axis of the frame, wherein each leaflet defines a cavity having an opening that is configured to face a direction of blood flow within the blood vessel, and wherein the inflow end of each leaflet is configured to move toward each other in response to a first increase in a blood pressure within the blood vessel, and in response to the blood pressure within the blood vessel being within a first blood pressure range.

Example 2: The flow restrictor of any example herein, in particular example 1, wherein the inflow end of each leaflet is configured to move toward each other in response to the blood pressure applying a first pressure to the leaflets within the pocket-like cavities.

Example 3: The flow restrictor of any example herein, in particular example 1, further comprising two or more springs, wherein the inflow end of each leaflet is coupled to the frame with one spring of the two or more springs.

Example 4: The flow restrictor of any example herein, in particular example 1, wherein the blood vessel is an inferior vena cava or a vein upstream from a renal vein.

Example 5: The flow restrictor of any example herein, in particular example 1, wherein the two or more leaflets are further configured to collapse or prolapse in response to a second increase in blood pressure within the blood vessel, and in response to the blood pressure within the blood vessel being within a second blood pressure range, wherein a lower limit of the second blood pressure range is higher than an upper limit of the first blood pressure range.

Example 6: The flow restrictor of any example herein, in particular example 5, wherein the first blood pressure range is associated with a pulmonary overload blood pressure, and the second blood pressure range is associated with an exertion-related blood pressure.

Example 7: The flow restrictor of any example herein, in particular example 5, wherein the first blood pressure range is from 10 mmHg to 25 mmHg and the second blood pressure range is greater than about 25 mmHg or greater than about 30 mmHg.

Example 8: The flow restrictor of any example herein, in particular example 5, wherein each leaflet collapsing or prolapsing comprises the inflow end of each leaflet moving farther away from each other.

Example 9: The flow restrictor of any example herein, in particular example 1, wherein each leaflet is relatively rigid compared to a coupling region at the outflow end of each leaflet, wherein the coupling region couples the outflow end of each leaflet to the frame, and wherein the coupling region is configured to flex to enable each leaflet to extend toward one another within the first blood pressure range.

Example 10: The flow restrictor of any example herein, in particular example 9, wherein the two or more leaflets are further configured to prolapse in response to the blood flow within a second blood pressure range, wherein the second blood pressure range is higher than the first blood pressure range, and wherein the coupling region is configured to flex to enable each leaflet to prolapse within the first blood pressure range.

Example 11: The flow restrictor of any example herein, in particular example 1, wherein each leaflet is relatively flexible compared to a coupling region at the outflow end of each leaflet, wherein the coupling region couples the outflow end of each leaflet to the frame, and wherein each leaflet is configured to flex to enable each leaflet to extend toward one another within the first blood pressure range.

Example 12: The flow restrictor of any example herein, in particular example 11, wherein the leaflets are further configured to prolapse in response to the blood flow within a second blood pressure range, wherein the second blood pressure range is higher than the first blood pressure range, and wherein each leaflet is configured to flex to enable each leaflet to prolapse within the first blood pressure range.

Example 13: The flow restrictor of any example herein, in particular example 1, wherein each leaflet is coupled to the frame using a flexible commissure, wherein the outflow end of each leaflet is coupled to an outflow portion of the flexible commissure, wherein the outflow portion of the flexible commissure is coupled to the frame, wherein the inflow end of each leaflet is coupled to an inflow portion of the flexible commissure, and wherein the inflow portion of the flexible commissure is configured to bend radially inward in response to the blood flow.

Example 14: A method of restricting blood flow within a blood vessel, comprising using the flow restrictor of any example herein, in particular any of examples 1-13, to restrict blood flow within the blood vessel.

Example 15: A method of treatment for a subject with congestive heart failure (CHF) and/or chronic kidney disease (CKD), comprising using the flow restrictor of any example herein, in particular any of examples 1-13, to restrict blood flow within the blood vessel.

Example 16: A bi-modal flow restrictor for a blood vessel, comprising: an outer frame positionable within a blood vessel; an inner valve positioned within the outer frame, wherein: the inner valve is movable in an axial direction within the outer frame, and the axial direction is approximately parallel with a central axis of the outer frame; two or more leaflets each comprising an inflow end and an outflow end, wherein: the outflow end of each leaflet is coupled to the inner valve, the inflow end of each leaflet is movable in a radial direction, the radial direction is approximately perpendicular to the central axis of the outer frame, each leaflet defines a cavity having an opening that is configured to face a direction of blood flow within the blood vessel, and the inflow end of each leaflet is configured to move toward each other in response to a first increase in a blood pressure within the blood vessel, and in response to the blood pressure within the blood vessel being within a first blood pressure range; and a first spring positioned between the inner valve and the outer frame, wherein: the first spring is configured to hold the inner valve in a first axial position within the outer frame when the blood pressure within the blood vessel is within the first blood pressure range, the first spring is configured to allow the inner valve to move to a second axial position when the blood pressure within the blood vessel is within a second blood pressure range, the second axial position is downstream from the first axial position, and a lower limit of the second blood pressure range is higher than an upper limit of the first blood pressure range; wherein an outflow ring of the inner valve is pressed against a seat of the outer frame when the inner valve is in the first axial position, and wherein the outflow ring of the inner valve is configured to move away from the seat of the outer frame when the inner valve is in the second axial position thereby allowing the blood flow to bypass the inner valve and flow between the inner valve and the outer frame.

Example 17: The bi-modal flow restrictor of any example herein, in particular example 16, wherein the first spring is in a first compression state when the inner valve is in the first axial position, wherein the first spring is in a second compression state when the inner valve is in the second axial position, and wherein the second compression state is a more compressed state than the first compression state.

Example 18: The bi-modal flow restrictor of any example herein, in particular example 16, wherein the inflow end of each leaflet is configured to move toward each other in response to the blood pressure applying a first pressure to the leaflets within the cavities.

Example 19: The bi-modal flow restrictor of any example herein, in particular example 16, further comprising two or more springs, wherein the inflow end of each leaflet is coupled to the frame with one spring of the two or more springs.

Example 20: The bi-modal flow restrictor of any example herein, in particular example 16, wherein the blood vessel is an inferior vena cava, or a vein upstream from a renal vein.

Example 21: The bi-modal flow restrictor of any example herein, in particular example 16, wherein the first blood pressure range is associated with a pulmonary overload blood pressure, and the second blood pressure range is associated with an exertion-related blood pressure.

Example 22: The bi-modal flow restrictor of any example herein, in particular example 16, wherein the first blood pressure range is from 10 mmHg to 25 mmHg and the second blood pressure range is greater than about 25 mmHg or greater than about 30 mmHg.

Example 23: The bi-modal flow restrictor of any example herein, in particular example 16, wherein each leaflet is coupled to the inner valve using a flexible commissure, wherein the outflow end of each leaflet is coupled to an outflow portion of the flexible commissure, wherein the outflow portion of the flexible commissure is coupled to the inner valve, wherein the inflow end of each leaflet is coupled to an inflow portion of the flexible commissure, and wherein the inflow portion of the flexible commissure is configured to bend radially inward in response to the blood flow.

Example 24: A method of restricting blood flow within a blood vessel, comprising using the bi-modal flow restrictor of any example herein, in particular in any of examples 16-23 to restrict blood flow within the blood vessel.

Example 25: A method of treating a patient with congestive heart failure (CHF) and/or chronic kidney disease (CKD), comprising using the bi-modal flow restrictor of any example herein, in particular any of examples 16-23, to restrict blood flow within the blood vessel.

Example 26: A bi-modal flow restrictor for a blood vessel, comprising: an outer docking frame positionable within a blood vessel; and an inner valve positioned within the outer docking frame, wherein, the inner valve is configured to be sealed against the outer docking frame when blood pressure within the blood vessel is within a first blood pressure range; wherein the inner valve comprises two or more leaflets configured to move closer to each other in response to elevated pressure within the blood vessel and restrict flow through the inner valve when the inner valve is sealed against the outer docking frame, wherein, the inner valve and the outer docking frame are configured such that a gap is formed between the inner valve and the outer docking frame when blood pressure within the blood vessel is within a second blood pressure range, wherein a lower limit of the second blood pressure range is higher than an upper limit of the first blood pressure range, and wherein blood can flow through the gap between the inner valve and the outer docking frame when the gap is formed.

Example 27: The bi-modal flow restrictor of any example herein, in particular example 26, wherein each of the two or more leaflets comprise an inflow end and an outflow end, wherein the outflow end of each leaflet is coupled to the inner valve, wherein the inflow end of each leaflet is movable in a radial direction, wherein the radial direction is approximately perpendicular to a central axis of the outer docking frame, wherein each leaflet defines a cavity having an opening that is configured to face blood flow within the blood vessel, and wherein, when the inner valve is sealed to the outer docking frame, the inflow end of each leaflet is configured to move toward each other in response to a first increase in a blood pressure within the blood vessel.

Example 28: The bi-modal flow restrictor of any example herein, in particular example 27, wherein the inflow end of each leaflet is configured to move toward each other in response to the blood pressure applying a first pressure to the leaflets within the cavities.

Example 29: The bi-modal flow restrictor of any example herein, in particular example 27, further comprising two or more springs, wherein the inflow end of each leaflet is coupled to the outer docking frame with one spring of the two or more springs.

Example 30: The bi-modal flow restrictor of any example herein, in particular example 27, wherein each leaflet is coupled to the inner valve using a flexible commissure, wherein the outflow end of each leaflet is coupled to an outflow portion of the flexible commissure, wherein the outflow portion of the flexible commissure is coupled to the inner valve, wherein the inflow end of each leaflet is coupled to an inflow portion of the flexible commissure, and wherein the inflow portion of the flexible commissure is configured to bend radially inward in response to the blood flow.

Example 31: The bi-modal flow restrictor of any example herein, in particular example 26, wherein the inner valve further comprises an inflow support portion pressing on an inflow side of a seat of the outer docking frame, wherein the inner valve further comprises an outflow portion pressing on an outflow side of the seat of the outer docking frame, wherein, when the blood pressure within the blood vessel is within the first blood pressure range, the inflow support portion and the outflow portion are configured to hold the inner valve in a first axial position within the outer docking frame, wherein, when the blood pressure within the blood vessel is within the second blood pressure range, the inflow support portion is configured to allow the inner valve to move to a second axial position downstream from the first axial position, thereby allowing the blood flow to bypass the inner valve and flow between the inner valve and the outer docking frame.

Example 32: The bi-modal flow restrictor of any example herein, in particular example 31, wherein the inflow support portion is in a first compression state when the inner valve is in the first axial position, wherein the inflow support portion is in a second compression state when the inner valve is in the second axial position, and wherein the second compression state is a more compressed state than the first compression state.

Example 33: The bi-modal flow restrictor of any example herein, in particular example 31, wherein the inner valve comprises an outer portion that comprises a valve cover, wherein the frame comprises a frame cover, and wherein the valve cover and the frame cover are configured to block blood flow from flowing between the inner valve and the frame when the outflow portion is pressed against the docking frame

Example 34: The bi-modal flow restrictor of any example herein, in particular example 26, wherein the blood vessel is an inferior vena cava, or a vein upstream from a renal vein.

Example 35: The bi-modal flow restrictor of any example herein, in particular example 26, wherein the first blood pressure range is associated with a pulmonary overload blood pressure, and the second blood pressure range is associated with an exertion-related blood pressure.

Example 36: The bi-modal flow restrictor of any example herein, in particular example 26, wherein the first blood pressure range is from 10 mmHg to 25 mmHg and the second blood pressure range is greater than about 25 mmHg or greater than about 30 mmHg.

Example 37: A method of restricting blood flow within a blood vessel, comprising using the bi-modal flow restrictor of any example herein, in particular any of examples 26-36, to restrict blood flow within the blood vessel.

Example 38: A method of treatment for a subject with congestive heart failure (CHF) and/or chronic kidney disease (CKD), comprising using the bi-modal flow restrictor of any of claims 26-36 to restrict blood flow within the blood vessel.

Example 39: A flow restrictor for a blood vessel, comprising: a frame positionable within a blood vessel; a first flap comprising a first flap inflow end and a first flap outflow end, wherein the first flap inflow end is coupled to the frame such that the first flap inflow end is movable in a radial direction, wherein the radial direction is approximately perpendicular to a central axis of the frame; a first control element coupled to the first flap; a first helical tube coupled to a wall of the blood vessel; and a first guide tube coupled to the frame, wherein the first control element is fixed to a first end of the first helical tube, extends through the first helical tube, exits the first helical tube at a second end, passes through the first guide tube, and is fixed to the first flap outflow end, wherein the first helical tube is configured to expand in response to an enlargement of the blood vessel, and wherein the first control element is configured to pull the first flap radially inward in response to the expansion of the first helical tube.

Example 40: The flow restrictor of any example herein, in particular example 39, wherein an open area defined by the blood vessel is configured to be narrowed upon the first flap moving radially inwards, and wherein a blood pressure downstream from the first flap is lowered when the open area defined by the blood vessel is narrowed.

Example 41: The flow restrictor of any example herein, in particular example 39, wherein the blood vessel enlarges due to elevated blood pressure due to one or more of: an increased blood pressure, a pulmonary overload, or an increased blood volume in the blood vessel.

Example 42: The flow restrictor of any example herein, in particular example 39, wherein the frame is expandable.

Example 43: The flow restrictor of any example herein, in particular example 39, wherein the blood vessel is an inferior vena cava, or in a vein upstream from a renal vein.

Example 44: The flow restrictor of any example herein, in particular example 39, wherein the first flap inflow end is coupled to the frame using a first pivotable coupling.

Example 45: The flow restrictor of any example herein, in particular example 39, further comprising: a second flap comprising a second flap inflow end and a second flap outflow end, wherein the second flap inflow end is coupled to the frame such that the second flap inflow end is movable in a radial direction; a second control element coupled to the second flap; a second helical tube coupled to a wall of the blood vessel; a second guide tube coupled to the frame; a third flap comprising a third flap inflow end and a third flap outflow end, wherein the third flap inflow end is coupled to the frame such that the third flap inflow end is movable in a radial direction; a third control element coupled to the third flap; a third helical tube coupled to a wall of the blood vessel; and a third guide tube coupled to the frame; wherein the first control element is fixed to a first end of the second helical tube, extends through the second helical tube, exits the second helical tube at a second end, passes through the second guide tube, and is fixed to the second flap outflow end, wherein the second helical tube is configured to expand in response to an enlargement of the wall of the blood vessel, wherein the second control element is configured to pull the second flap radially inward in response to the expansion of the first helical tube, wherein the third control element is fixed to a first end of the third helical tube, extends through the third helical tube, exits the third helical tube at a second end, passes through the third guide tube, and is fixed to the third flap outflow end, wherein the third helical tube is configured to expand in response to an enlargement of the wall of the blood vessel, and wherein the third control element is configured to pull the third flap radially inward in response to the expansion of the first helical tube.

Example 46: The flow restrictor of any example herein, in particular example 39, wherein the first flap is further configured to prolapse in response to the blood vessel expanding beyond a first threshold.

Example 47: The flow restrictor of any example herein, in particular example 46, wherein the blood vessel enlarging beyond the first threshold is associated with an exertion-related blood pressure.

Example 48: The flow restrictor of any example herein, in particular example 46, wherein the first flap prolapsing comprises the outflow end of the first flap moving axially towards an inflow side of the frame.

Example 49: The flow restrictor of any example herein, in particular example 39, wherein the first flap inflow end is coupled to the frame using a first pivotable coupling.

Example 50: A method of restricting blood flow within a blood vessel, comprising using the flow restrictor of any example herein, in particular any of examples 39-49, to restrict blood flow within the blood vessel.

Example 51: A method of treatment for a subject with congestive heart failure (CHF) and/or chronic kidney disease (CKD), comprising using the flow restrictor of any example herein, in particular any of examples 39-49, to restrict blood flow within the blood vessel.

Example 52: A flow restrictor for a blood vessel, comprising: a frame configured to be coupled to a wall of a blood vessel, wherein a cross-section of the frame approximately perpendicular to a central axis of the frame is approximately oval-shaped; a first flap coupled to an outflow end of the frame using a first coupling such that the first flap is movable about the first coupling; and a first member coupled to an inflow end of the frame and to the first flap, wherein the first member has an approximately constant length, wherein, in response to an increase in blood pressure, the cross-sectional area of the frame is configured to increase by assuming a less oval cross-sectional shape approximately perpendicular to the central axis of the frame, and wherein the first member is configured to move the first flap in response to the frame assuming the less oval cross-sectional shape.

Example 53: The flow restrictor of any example herein, in particular example 52, wherein the first member is a rigid push member, or a flexible pull member.

Example 54: The flow restrictor of any example herein, in particular example 52, wherein the first member is a rigid rod, or a flexible wire.

Example 55: The flow restrictor of any example herein, in particular example 52, further comprising a second member coupled to the frame and the first flap, wherein the first member and the second member are configured to move the first flap together, in response to the frame assuming the less oval cross-sectional shape.

Example 56: The flow restrictor of any example herein, in particular example 52, wherein, in response to the blood pressure rising above a threshold, a change in the cross-sectional shape of the frame is limited, and wherein the blood pressure above the threshold causes the blood vessel to expand such that channels are formed between the frame and the blood vessel through which blood can flow.

Example 57: The flow restrictor of any example herein, in particular example 56, wherein the blood pressure rising above the threshold is associated with an exertion-related blood pressure within the blood vessel.

Example 58: The flow restrictor of any example herein, in particular example 52, wherein, the frame is oversized relative to the blood vessel, and wherein, in response to the blood pressure rising above a threshold, the frame and blood vessel assume an approximately circular cross-sectional shape approximately perpendicular to the central axis of the frame.

Example 59: The flow restrictor of any example herein, in particular example 58, wherein the blood pressure rising above the threshold is associated with an exertion-related blood pressure within the blood vessel.

Example 60: The flow restrictor of any example herein, in particular example 52, wherein a long axis of the oval-shaped cross-section at the outflow end of the frame intersects the frame in a long-axis region, and wherein the first flap is coupled to the outflow end of the frame at the long-axis region.

Example 61: The flow restrictor of any example herein, in particular example 52, wherein a short axis of the oval-shaped cross-section at the outflow end of the frame intersects the frame in a short-axis region, and wherein the first flap is coupled to the outflow end of the frame at the short-axis region.

Example 62: The flow restrictor of any example herein, in particular example 52, wherein the flow restrictor is configured to restrict blood flow within the blood vessel by different amounts in response to the increase in blood pressure being within different pressure ranges.

Example 63: The flow restrictor of any example herein, in particular example 52, further comprising: a second flap coupled to the outflow end of the frame using a first coupling such that the first flap is movable about the coupling, wherein the second flap is coupled to the outflow end of the frame on an approximately opposite side of the frame; and a second member coupled to an inflow end of the frame and to the second flap, wherein the second member has an approximately constant length, and wherein the second member is coupled to the inflow end of the frame on an approximately opposite side of the frame as the first flap, wherein the second member is configured to move the second flap in response to the frame assuming the less oval cross-sectional shape.

Example 64: The flow restrictor of any example herein, in particular example 52, wherein: the first member is a rigid rod, the first flap comprises an outer edge and an inner edge approximately opposite to the inner edge, a long axis of the oval-shaped cross-section at the outflow end of the frame intersects the frame in a first long-axis region, the outer edge first flap is coupled to the outflow end of the frame at the first long axis region, the long axis of the oval-shaped cross-section at the inflow end of the frame intersects the frame in a second long-axis region that is opposite the first long-axis region, the rigid rod is coupled to the inflow end of the frame at the second long axis region, and in response to the frame assuming a less oval cross-sectional shape, the inner end of the flap moves axially towards the outflow direction.

Example 65: The flow restrictor of any example herein, in particular example 64, wherein a geometry of the frame and the first member are configured to cause the first flap to be angled towards the inflow end of the frame at lower blood pressures, and to move axially towards the outflow end of the frame as the blood pressure increases.

Example 66: The flow restrictor of any example herein, in particular example 52, wherein: the first member is a first flexible wire, the first flap comprises an outer edge and an inner edge approximately opposite to the inner edge, a long axis of the oval-shaped cross-section at the outflow end of the frame intersects the frame in a first long-axis region, the outer edge first flap is coupled to the outflow end of the frame at the first long-axis region, the long axis of the oval-shaped cross-section at the inflow end of the frame intersects the frame in a second long-axis region that is opposite the first long-axis region, the first flexible wire is coupled to the inflow end of the frame at the second long-axis region, and wherein, in response to the frame assuming a less oval cross-sectional shape, the inner end of the flap moves axially towards the outflow direction.

Example 67: The flow restrictor of any example herein, in particular example 66, wherein a geometry of the frame and the first member are configured to cause the first flap to be angled towards the outflow end of the frame at lower blood pressures, in response to the blood flow within the blood vessel, and to move axially towards the inflow end of the frame as the blood pressure increases.

Example 68: The flow restrictor of any example herein, in particular example 66, further comprising a second member coupled to the first flap and the frame, wherein the second member is a second flexible wire, and wherein the first member and the second member are configured to move the first flap together, in response to the frame assuming a less oval cross-sectional shape.

Example 69: A method of restricting blood flow within a blood vessel, comprising using the flow restrictor of any example herein, in particular any of examples 52-68, to restrict blood flow within the blood vessel.

Example 70: A method of treatment for a subject with congestive heart failure (CHF) and/or chronic kidney disease (CKD), comprising using the flow restrictor of any example herein, in particular any of examples 52-68, to restrict blood flow within the blood vessel.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

The systems and methods of the embodiments and variations described herein can be embodied and/or implemented at least in part as a machine configured to receive a computer-readable medium storing computer-readable instructions. The instructions may be executed by computer-executable components integrated or in communication with the system and one or more portions of the processor on or in communication with any device described herein (e.g., a flow restrictor of FIGS. 1-28A) and/or computing device. For example, the flow restrictors described herein may be controllable by a control device such that the control device includes computer-executable components configured to execute the instructions. The flow restrictors may be in wireless or wired (e.g., via a lead) communication with the control device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “flap” or “leaflet” may include, and is contemplated to include, a plurality of flaps or leaflets. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, may be apparent to those of skill in the art upon reviewing the above description.

What is claimed is:

1. A flow restrictor for a blood vessel, comprising:
a frame positionable within the blood vessel; and
two or more leaflets each comprising an inflow end and an outflow end,
wherein the outflow end of each leaflet is coupled to the frame,
wherein the inflow end of each leaflet is movable in a radial direction, wherein the radial direction is approximately perpendicular to a central axis of the frame,
wherein each leaflet defines a cavity having an opening that is configured to face a direction of blood flow within the blood vessel, and
wherein the inflow end of each leaflet is configured to move toward each other in response to a first increase in a blood pressure within the blood vessel, and in response to the blood pressure within the blood vessel being within a first blood pressure range.

2. The flow restrictor of claim 1, wherein the inflow end of each leaflet is configured to move toward each other in response to the blood pressure applying a first pressure to the leaflets within the cavities.

3. The flow restrictor of claim 1, wherein the blood vessel is an inferior vena cava or a vein upstream from a renal vein.

4. The flow restrictor of claim 1, wherein the two or more leaflets are further configured to collapse or prolapse in response to a second increase in the blood pressure within the blood vessel, and in response to the blood pressure within the blood vessel being within a second blood pressure range, wherein a lower limit of the second blood pressure range is higher than an upper limit of the first blood pressure range.

5. The flow restrictor of claim 4, wherein the first blood pressure range is from 10 mmHg to 25 mmHg and the second blood pressure range is greater than about 25 mmHg or greater than about 30 mmHg.

6. The flow restrictor of claim 4, wherein each leaflet collapsing or prolapsing comprises the inflow end of each leaflet moving farther away from each other.

7. The flow restrictor of claim 1, wherein each leaflet is coupled to the frame using a flexible commissure, wherein the outflow end of each leaflet is coupled to an outflow portion of the flexible commissure, wherein the outflow portion of the flexible commissure is coupled to the frame, wherein the inflow end of each leaflet is coupled to an inflow portion of the flexible commissure, and wherein the inflow portion of the flexible commissure is configured to bend radially inward in response to the blood flow.

8. A flow restrictor for a blood vessel, comprising:
a frame positionable within the blood vessel; and
two or more leaflets each comprising an inflow end and an outflow end,
wherein the outflow end of each leaflet is coupled to the frame,
wherein the inflow end of each leaflet is movable in a radial direction, wherein the radial direction is approximately perpendicular to a central axis of the frame, and
wherein the inflow end of each leaflet is configured to move toward each other in response to a first increase in a blood pressure within the blood vessel, and in response to the blood pressure within the blood vessel being within a first blood pressure range.

9. The flow restrictor of claim 8, wherein each leaflet defines a cavity having an opening, and
wherein the inflow end of each leaflet is configured to move toward each other in response to the blood pressure applying a first pressure to the leaflets within the cavities.

10. The flow restrictor of claim 8, wherein the blood vessel is an inferior vena cava or a vein upstream from a renal vein.

11. The flow restrictor of claim 8, wherein the two or more leaflets are further configured to collapse or prolapse in response to a second increase in the blood pressure within the blood vessel, and in response to the blood pressure within the blood vessel being within a second blood pressure range, wherein a lower limit of the second blood pressure range is higher than an upper limit of the first blood pressure range.

12. The flow restrictor of claim 11, wherein the first blood pressure range is from 10 mmHg to 25 mmHg and the second blood pressure range is greater than about 25 mmHg or greater than about 30 mmHg.

13. The flow restrictor of claim 11, wherein each leaflet collapsing or prolapsing comprises the inflow end of each leaflet moving farther away from each other.

14. The flow restrictor of claim 8, wherein each leaflet is coupled to the frame using a flexible commissure, wherein the outflow end of each leaflet is coupled to an outflow portion of the flexible commissure, wherein the outflow portion of the flexible commissure is coupled to the frame, wherein the inflow end of each leaflet is coupled to an inflow portion of the flexible commissure, and wherein the inflow portion of the flexible commissure is configured to bend radially inward in response to a blood flow in the blood vessel.

15. A flow restrictor for a blood vessel, comprising:
a frame positionable within the blood vessel; and
two or more leaflets each comprising an inflow end and an outflow end,
wherein the outflow end of each leaflet is coupled to the frame,
wherein the inflow end of each leaflet is movable in a radial direction, wherein the radial direction is approximately perpendicular to a central axis of the frame, and
wherein each leaflet defines a cavity having an opening that is configured to face a direction of blood flow within the blood vessel.

16. The flow restrictor of claim 15, wherein the inflow end of each leaflet is configured to move toward each other in response to a first increase in a blood pressure within the blood vessel, and
wherein the inflow end of each leaflet is configured to move toward each other in response to the blood pressure applying a first pressure to the leaflets within the cavities.

17. The flow restrictor of claim 15, wherein the blood vessel is an inferior vena cava or a vein upstream from a renal vein.

18. The flow restrictor of claim 16, wherein the two or more leaflets are further configured to collapse or prolapse in response to a second increase in the blood pressure within the blood vessel, and in response to the blood pressure within the blood vessel being within a second blood pressure range, wherein a lower limit of the second blood pressure range is higher than an upper limit of a first blood pressure range.

19. The flow restrictor of claim 18, wherein the first blood pressure range is from 10 mmHg to 25 mmHg and the second blood pressure range is greater than about 25 mmHg or greater than about 30 mmHg.

20. The flow restrictor of claim 18, wherein each leaflet collapsing or prolapsing comprises the inflow end of each leaflet moving farther away from each other.
